(19) United States
(12) Patent Application Publication
(10) Pub. No.: US 2024/0122994 A1
(43) Pub. Date: Apr. 18, 2024

(54) AMMONIA OXIDIZING MICROORGANISMS FOR USE AND DELIVERY TO THE GASTROINTESTINAL SYSTEM

(71) Applicant: AOBIOME LLC, Cambridge, MA (US)

(72) Inventors: David R. Whitlock, Cambridge, MA (US); Larry Weiss, San Francisco, CA (US); Lauren Nicole Ambrogio, Boulder, CO (US)

(21) Appl. No.: 18/391,604

(22) Filed: Dec. 20, 2023

Related U.S. Application Data
(63) Continuation of application No. 18/329,104, filed on Jun. 5, 2023.
Application No. 18/329,104 is a continuation of application No. 18/050,780, filed on Oct. 28, 2022, now abandoned.
Application No. 18/050,780 is a continuation of application No. 17/660,113, filed on Apr. 21, 2022, now abandoned.
Application No. 17/660,113 is a continuation of application No. 17/497,332, filed on Oct. 8, 2021, now abandoned.
Application No. 17/497,332 is a continuation of application No. 17/221,104, filed on Apr. 2, 2021, now abandoned.
Application No. 17/221,104 is a continuation of application No. 16/318,583, filed on Mar. 19, 2019, now abandoned.
(60) Provisional application No. 62/364,079, filed on Jul. 19, 2016.

(51) Int. Cl. A61K 35/741; A23L 33/135; A61K 9/00; A61K 9/02; A61K 9/08; A61K 33/00; A61K 35/74; A61K 45/06; A61K 47/02

(52) U.S. Cl. CPC A61K 35/741; A23L 33/135; A61K 9/0031; A61K 9/0043; A61K 9/0053; A61K 9/0056; A61K 9/006; A61K 9/0073; A61K 9/02; A61K 9/08; A61K 33/00; A61K 35/74; A61K 45/06; A61K 47/02; A23V 2002/00; A61K 2035/115; Y02A 50/30

(57) ABSTRACT

Ammonia oxidizing microorganism preparations for delivery to the gastrointestinal system, kits including ammonia oxidizing preparations for delivery to the gastrointestinal system, and devices for administering ammonia oxidizing preparations to the gastrointestinal system are provided. Methods of introducing ammonia oxidizing microorganisms to the gastrointestinal system are provided. Methods of treating disorders, including gastrointestinal disorders and inflammatory disorders, with ammonia oxidizing microorganism preparations are provided.

### CROSS-REFERENCE TO RELATED APPLICATIONS

This application is a continuation of U.S. patent application Ser. No. 18/329,104, which is a continuation of U.S. patent application Ser. No. 18/050,780, which is a continuation of U.S. patent application Ser. No. 17/660,113, which is a continuation of U.S. patent application Ser. No. 17/497,332, which is a continuation of U.S. patent application Ser. No. 17/221,104, which is a continuation of U.S. patent application Ser. No. 16/318,583, which is a U.S. national phase application, and claims the benefit of priority under 35 U.S.C. § 371, of International (PCT) Patent Application Serial No. PCT/US2017/042622, filed on Jul. 18, 2017, which in turn claims priority under 35 U.S.C. § 119(e) to U.S. Provisional Patent Application Ser. No. 62/364,079 as filed on Jul. 19, 2016, as well as to U.S. Provisional Patent Application Ser. No. 62/397,710 as filed on Sep. 21, 2016, the entire disclosure of each of which is hereby incorporated herein by reference in its entirety for all purposes.

### FIELD OF THE TECHNOLOGY

Aspects relates generally to the microbiome and, more specifically, to the restoration of ammonia oxidizing microorganisms in relation to the microbiome.

### BACKGROUND

Bacteria and other microorganisms are ubiquitous in the environment. The discovery of pathogenic bacteria and the germ theory of disease have had a tremendous effect on health and disease states. Microorganisms are a normal part of the environment of all living things and may be beneficial. In the gut, for example, bacteria are not pathogenic under normal conditions, and in fact improve health by rendering the normal intestinal contents less hospitable for disease causing organisms.

### SUMMARY

In accordance with one or more aspects, a method of introducing ammonia oxidizing microorganisms (AOM) to a subject is disclosed. The method may comprise enterally administering a preparation comprising AOM to the subject.

In some aspects, enteral administration may comprise oral, buccal, sublabial, or sublingual administration.

In accordance with one or more aspects, a method of introducing ammonia oxidizing microorganisms (AOM) to a subject is disclosed. The method may comprise rectally administering a preparation comprising AOM to the subject.

In some aspects, rectal administration may comprise administration via suppository or enema. In other aspects, rectal administration may be associated with a fecal microbiota transplant procedure.

In accordance with one or more aspects, a method of colonizing a gastrointestinal system of a subject is disclosed. The method may comprise administering an effective amount of a preparation comprising AOM to the gastrointestinal system of the subject, wherein the AOM colonize a target tissue of the gastrointestinal system.

In accordance with one or more aspects, a method of improving digestion in a subject is disclosed. The method may comprise administering to the subject an effective amount of a preparation comprising AOM, thereby improving digestion in the subject.

In accordance with one or more aspects, a method of treating a gastrointestinal disorder in a subject is disclosed. The method may comprise administering to the subject an effective amount of a preparation comprising AOM, thereby treating the gastrointestinal disorder.

In some aspects, a target percentage of administered AOM are transferred to a gastrointestinal system of the subject. An amount and/or a frequency of administration is sufficient to increase mucus thickness in at least a portion of the gastrointestinal system of the subject. Administering the preparation may result in increased tolerance, decreased sensitivity, and/or improved uptake of nutrients in connection with a food or a beverage ingested by the subject.

In at least some aspects, the preparation comprising AOM is administered via ingestion to a gastrointestinal system of a subject. The subject may have a substantially empty stomach when the preparation is administered. The preparation may be administered subsequent to administration of an antibiotic or a cleansing bowel preparation. he method may further comprise administering water to the subject subsequent to administering the preparation.

In some aspects, the gastrointestinal disorder may be an inflammatory condition. For example, the inflammatory condition may be colitis, necrotizing enterocolitis, inflammatory bowel disease, ulcer, Crohn's Disease, ulcerative colitis, Celiac's Disease, gluten sensitivity, heartburn, pancreatitis, appendicitis, gastritis, gastroenteritis, irritable bowel syndrome, or a dental or periodontal condition. The inflammatory condition may be associated with catheter-based delivery of a substance, e.g. enteral nutrition. The inflammatory condition may be an infection by one or more of the following microorganisms: H. pylori, C. diff, cholera, amoebic dysentery, Y. enterocolitica, S. enteritidis, S. typhimurium, Shigella sonnei, and E. coli. In other aspects, the gastrointestinal disorder may relate to a lactose, food, or beverage intolerance, SIBO, a malabsorption disorder, a biliary disorder, reflux, or dispoxia. The gastrointestinal disorder may be characterized by constipation or diarrhea. The gastrointestinal disorder is characterized by hyperammonemia. The gastrointestinal disorder may comprise liver failure, e.g. acute or chronic liver failure.

In some aspects, administration may reduce bloating, diarrhea, gas, stomach pain, stomach cramping, or borborygmus in the subject.

In some aspects, administration may precede or follow a medical procedure, e.g., a catheterization, endoscopy, or colonoscopy procedure, or a dental procedure. Administration may be device-assisted. The preparation may be administered prior to onset, during incidence, or subsequent to the subsiding of a gastrointestinal condition. The preparation may be administered in response to a gastrointestinal symptom, trigger or warning sign, e.g. discomfort or a change in bowel habit. A method may involve determining if the subject is in need of treatment for a gastrointestinal disorder.

In some aspects, administration may be to a deposit tissue or directly to a target tissue. A deposit tissue, target tissue, or both may be a mucous membrane of the subject. The deposit tissue, target tissue, or both may be associated with a stomach of the subject. The deposit tissue, target tissue, or both may be a salivary gland, oral cavity, pharynx, tongue, esophagus, liver, gallbladder, common bile duct, colon (transverse, ascending, and/or descending), cecum, appendix, rectum, anus, pancreas, pancreatic duct, large intestine, or small intestine (duodenum, jejunum, and/or ileum) of the subject.

In some aspects, the target tissue may be associated with a desired local effect. The target tissue may be associated with a desired systemic effect. For example, a desired systemic effect may involve treatment of one or more of: headaches, cardiovascular diseases, inflammation, immune responses, autoimmune disorders, liver diseases, infections, neurological diseases, psychiatric disorders, nitric oxide disorders, urea cycle disorders, congestion, vasodilation disorders, skin diseases, wound healing, reactions to insect bites, ophthalmic disorders, connective tissue disorders, and certain viral, bacterial, or fungal infections.

In some aspects, administering an effective amount of the preparation may promote endothelial function. Administering an effective amount of the preparation may change or alter a level of nitrite or NO at a target tissue or in circulation. Administering an effective amount of the preparation may modulate a microbiome associated with the gastrointestinal system of the subject.

In some aspects, the preparation may be administered as a solution, suspension, emulsion, ointment, gel, hydrogel, or liquid, e.g. drop, spray, aerosol, or mist. The preparation may be formulated as a tablet or capsule. The preparation may include microspheres or microcapsules. The preparation may be formulated to be compatible with the gastrointestinal system of the subject. The preparation may be formulated for immediate release or extended release. The preparation may be formulated to deliver nitrite or NO to a target tissue, locally or systemically. The preparation may be formulated for transmucosal delivery and/or circulation, e.g. locally or systemically.

In some aspects, a treatment method may further comprise administering a second amount of the preparation to the subject. In at least some aspects, the preparation may be administered as part of a combination therapy. The method may further comprise administering a second treatment in combination with the preparation. The preparation may be administered for a period of time prior to initiating the second treatment, concurrently with the second treatment, or for a period of time subsequent to ceasing the second treatment. The second treatment may be administered via an alternate mode of administration, e.g. via inhalation or intranasal technique. The subject may have a therapeutic level of a second treatment. The preparation may be administered in conjunction with an anti-inflammatory agent. The preparation may be administered in conjunction with a medical approach that treats, e.g., is approved to treat or is commonly used to treat, the relevant disease or disorder, or a symptom of the relevant disease or disorder. The preparation may be administered before or after a surgical or diagnostic procedure. The second treatment may comprise a surgical procedure. The preparation may be administered in conjunction with exercise, fiber, laxative, antidiuretic, probiotic, therapeutic, or stress management. In at least some aspects, the preparation may be administered in combination with a therapeutic treatment for colitis, necrotizing enterocolitis, inflammatory bowel disease, ulcer, Crohn's Disease, ulcerative colitis, Celiac's Disease, gluten sensitivity, heartburn, pancreatitis, appendicitis, gastritis, gastroenteritis, or irritable bowel syndrome. The preparation may be administered in conjunction with nitrite, nitrate, and/or NO.

In some aspects, the effective amount is a therapeutically effective dose of AOM. The therapeutically effective dose of AOM is about or greater than about 1×10³, 10⁴, 10⁵, 10⁶, 10⁷, 10⁸, 10⁹, 10¹⁰, 10¹¹, 10¹², 10¹³, or 10¹⁴ CFU. The preparation may be administered as an analgesic and/or as a prophylactic. The preparation may be self-administered. The preparation may be administered about 1, 2, 3, 4, 5, 6, 7, 8, 9, 10, 11, 12, 13, 14, 15, 16, 17, 18, 19, 20, 21, 22, 23, or 24 times per day. The preparation may be administered for about 1-3, 3-5, 5-7, 7-9, 5-10, 10-14, 12-18, 12-21, 21-28, 28-35, 35-42, 42-49, 49-56, 46-63, 63-70, 70-77, 77-84, or 84-91 days. The preparation may be administered within 30, 60, 90, 120, 150, or 180 minutes of the subject waking from sleep. The preparation may be administered within 30, 60, 90, 120, 150, or 180 minutes prior to the subject sleeping. The preparation may be administered within 30, 60, 90, 120, 150, or 180 minutes of the subject eating. The preparation may be administered 30, 60, 90, 120, 150, or 180 minutes before the subject cleanses or showers.

In some aspects, the subject may be female. In other aspects, the subject may be male. The subject may be characterized as one of the following ethnicity/race: Asian, black or African American, Hispanic or Latino, white, or multi-racial. The subject may have a disrupted microbiome. The subject may be of an age less than 1, or between 1-5, 5-10, 10-20, 20-30, 30-40, 40-50, 50-60, or over 60 years.

In some aspects, the preparation may comprise AOM in a buffer solution, e.g., an aqueous buffer solution. The buffer solution, e.g., aqueous buffer solution, comprises disodium phosphate and magnesium chloride, for example, 50 mM Na₂HPO₄ and 2 mM MgCl₂ in water. The buffer solution e.g., aqueous buffer solution, consisting essentially of disodium phosphate and magnesium chloride, for example, 50 mM Na₂HPO₄ and 2 mM MgCl₂ in water. The buffer solution, e.g., aqueous buffer solution, consists of disodium phosphate and magnesium chloride, for example, 50 mM Na₂HPO₄ and 2 mM MgCl₂ in water. The preparation may be characterized by a physiological pH level. The preparation may further comprise or be administered concurrently with a compound that promotes growth or metabolism of the AOM, NO production, and/or urease activity. In at least some aspects, the preparation may comprise at least one of ammonia, ammonium salts, and urea. The preparation may comprise a controlled release material, e.g., slow release material. The preparation may further comprise an excipient, e.g., a pharmaceutically acceptable excipient. The excipient may comprise an absorption or penetration enhancer, preservative, antioxidant, buffer, chelating agent, ion exchange agent, solubilizing agent, suspending agent, thickener, surfactant, wetting agent, tonicity-adjusting agent, enzyme inhibitor, or vehicle for proper drug delivery. The excipient may comprise an anti-adherent, binder, coat, disintegrant, filler, flavor, color, lubricant, glidant, sorbent preservative, or sweetener. In at least some aspects, the preparation may comprise a mucoadhesive agent, disintegrant, chelator, coating agent, modified-release product, or filler. The preparation may be substantially free of other organisms. The preparation may further comprise other organisms, e.g., a community of organisms.

In some aspects, the preparation may comprise between about 1×10³ CFU/mL to about 1×10¹⁴ CFU/mL AOM. The preparation may comprise between about 1×10⁹ CFU/mL to about 10×10⁹ CFU/mL AOM. The AOM may comprise ammonia oxidizing bacteria (AOB). In some aspects, the AOM may consist essentially of AOB. In at least some aspects, the AOM may consist of AOB. The AOM may comprise Nitrosomonas, Nitrosococcus, Nitrosospira, Nitrosocystis, Nitrosolobus, Nitrosovibrio, and combinations thereof. The AOM may be Nitrosomonas eutropha (N. eutropha). The AOM may be N. eutropha D23, having ATCC accession number PTA-121157. In at least some aspects, the AOM may comprise ammonia oxidizing archaea (AOA). The AOM may be capable of converting ammonia or ammonium to nitrite at a rate of at least about 1 pmol/min/mg protein. The AOM may be capable of converting ammonia or ammonium to nitrite at a rate of at least about 0.1 nmol/min/mg protein.

In some aspects, the preparation may be administered, e.g., via ingestion to a first tissue, e.g. a deposit tissue. The first tissue may be the target tissue. In at least some aspects, the first tissue is other than the target tissue, e.g., the preparation may be applied to a first tissue and the preparation, or a product of the preparation, e.g., NO, may be transported, e.g., by diffusion, to a second tissue, e.g. the target tissue.

In some aspects, a biome-friendly product may be used in connection with the administered preparation comprising AOM.

In accordance with one or more aspects, a preparation comprising AOM, as recited in any of the preceding claims, may be for enteral administration to a subject.

In some aspects, the preparation may be a food product. The food product may comprise a food, drink, supplement, nutraceutical, additive, or medical nutritional product.

In accordance with one or more aspects, a preparation may comprise AOM. The preparation may be for treatment of a gastrointestinal disorder in a subject.

In some aspects, the preparation may be packaged for single use. In some aspects, the preparation may be packaged for multiple use.

In accordance with one or more aspects, a device may be configured to administer a preparation comprising AOM to a target tissue of a gastrointestinal system of a subject.

In accordance with one or more aspects, a kit may comprise a preparation comprising AOM, e.g., for delivery to a gastrointestinal system of a subject or for treatment of a gastrointestinal disorder in a subject.

The disclosure contemplates all combinations of any one or more of the foregoing aspects and/or embodiments, as well as combinations with any one or more of the embodiments set forth in the detailed description and any examples.

### DETAILED DESCRIPTION

In accordance with one or more embodiments, the present disclosure provides for various methods or modes of introducing ammonia oxidizing microorganisms to a subject. These methods or modes comprise administering to a subject ammonia oxidizing microorganisms, for example, a preparation, composition, formulation, or product comprising ammonia oxidizing microorganisms. In at least some embodiments, ammonia oxidizing microorganisms may therefore generally be restored to a microbiome of the subject. In at least some embodiments, ammonia oxidizing microorganisms may comprise or consist essentially of live ammonia oxidizing microorganisms.

Preparations, compositions, and/or formulations, e.g., including cosmetic products, therapeutic products, consumer products, non-natural products, natural products, and fortified natural products, comprising, consisting essentially of, or consisting of ammonia oxidizing microorganisms are disclosed. These preparations, compositions, and/or formulations are disclosed herein for use in various applications, e.g., cosmetic and/or therapeutic applications. The preparations, compositions, and/or formulations may be administered in an effective amount for an intended use, e.g., a cosmetic or a therapeutic application. Preparations, compositions, and/or formulations comprising ammonia oxidizing microorganisms for various modes of administration to a subject are provided. Preparations, compositions, and/or formulations comprising ammonia oxidizing microorganisms for use in the treatment of various conditions and/or disorders in a subject are provided. Methods of treating a subject for various conditions and/or disorders via administration of ammonia oxidizing microorganisms are disclosed. Devices for use in administering ammonia oxidizing microorganisms to a subject are also provided.

### Microbiology

In accordance with one or more embodiments, essentially any ammonia oxidizing microorganism (AOM) can be used or implemented. The ammonia oxidizing microorganisms may generally be autotrophic. The ammonia oxidizing microorganisms may generate nitrite and/or nitric oxide from ammonia.

Properties of autotrophic ammonia oxidizing bacteria (AOB), for example, are well described by Whitlock in U.S. Pat. No. 7,820,420. Since that filing, the class of autotrophic microorganisms that oxidize ammonia for ATP production has been expanded to encompass ammonia oxidizing archaea (AOA), and archaea have been moved out of the class of bacteria and into their own distinct class. For the purposes of this disclosure, any and all autotrophic ammonia oxidizing microorganisms that share the properties of oxidation of ammonia to generate ATP can be implemented. AOM, including both AOB and AOA, share the necessary properties of oxidation of ammonia into NO and nitrite and all known AOM lack capacity for virulence because of their inability to use organic substrates for ATP generation. Bacteria can utilize ammonia at higher concentrations, while archaea can utilize ammonia at lower concentrations. Physiological levels of ammonia are within the range that both bacteria (AOB) and archaea (AOA) can utilize. Any reference specifically to ammonia oxidizing bacteria throughout this disclosure should be considered equally applicable to any ammonia oxidizing microorganism, e.g., any ammonia oxidizing archaea, and these terms may all be used interchangeably herein.

Ammonia oxidizing bacteria (AOB) are ubiquitous Gram-negative obligate bacteria with a unique capacity to generate energy exclusively from the conversion of ammonia to nitrite. In some embodiments, ammonia oxidizing bacteria (AOB) of the genus Nitrosomonas are Gram-negative obligate autotrophic (chemolithoautotrophic) bacteria with a unique capacity to generate nitrite and nitric oxide exclusively from ammonia as an energy source. They are widely present both in soil and water environments and are essential components of environmental nitrification processes. These bacteria have beneficial properties, e.g., in connection with various cosmetic and therapeutic uses, in accordance with one or more embodiments described herein. Without wishing to be bound to any particular theory, due to the roles of nitrite and nitric oxide as important components of several physiological functions, such as vasodilation, inflammation and wound healing, these bacteria may have various beneficial properties for both healthy and immunopathological conditions. These bacteria are safe for use in humans because they are slow-growing, cannot grow on organic carbon sources, may be sensitive to soaps and antibiotics, and have never been associated with any disease or infection in animals or humans.

Ammonia oxidizing microorganisms generate coenzyme Q 8 (CoQ8) as a byproduct of the process by which they generate nitrite and nitric oxide. CoQ8 is a coenzyme Q having 8 carbons in its isoprenoid side chain. Without wishing to be bound to any particular theory, due to the role of coenzyme Q as an important component of several cell functions, such as mediating cell signaling and preventing cell death (anti-aging), these microorganisms' beneficial properties may further be enhanced by their specific ability to generate CoQ8.

In some embodiments, ammonia oxidizing bacteria may catalyze the following reactions.

At a neutral pH level, ammonia generated from ammonium around neutral pH conditions is the substrate of the initial reaction. The conversion of ammonia to nitrite takes place in two steps catalyzed respectively by ammonia monooxygenase (AMO) and hydroxylamine oxidoreductase (HAO), as follows:

NH₃+2H⁺+2e−+O₂→NH₂OH+H₂O (A)

NH₂OH+H₂O→NO₂⁻+4e−+5H⁺ (B)

In some instances, reaction B is reported as follows, to indicate nitrous acid (HNO₂) formation at low pH:

NH₂OH+H₂O→HNO₂+4e−+4H⁺

In certain embodiments, NH₄⁺ and NH₃ may be used interchangeably throughout the disclosure.

Examples of ammonia oxidizing bacteria include Nitrosomonas eutropha strains, e.g., D23 and C91 as discussed herein, and other bacteria in the genera Nitrosomonas, Nitrosococcus, Nitrosospira, Nitrosocystis, Nitrosolobus, and Nitrosovibrio. D23 Nitrosomonas eutropha strain refers to the strain, designated AOB D23-100, deposited with the American Tissue Culture Collection (ATCC) (10801 University Blvd., Manassas, VA, USA) on Apr. 8, 2014 having accession number PTA-121157. The nucleic acid sequence(s), e.g., genome sequence, of accession number PTA-121157 are hereby incorporated herein by reference in their entireties for all purposes. “AOB D23-100” may also be referred to as D23 or B244 throughout this disclosure.

Examples of ammonia oxidizing archaea include archaea in the genera Methanobrevibacter, Methanosphaera, Methanosarcina, Nitroscaldus, Nitrosopumilus, and Nitrososphaera (e.g. Nitrososphaera viennensis, Nitrososphaera gargensis). Different phylotypes of archaea, e.g., methanogens and halphilic archaeon, may be included in the preparations disclosed herein. Examples of archaea further include archaea in the lineages of phyla Euryarchaeota (e.g. Methanosarcina), Crenarchaeota, Aigarchaeota, and Thaumarchaeota (e.g. Giganthauma karukerense, Giganthauma insulaporcus, Caldiarchaeum subterraneum, Cenarchaeum symbiosum).

Each and every nucleic acid sequence and amino acid sequence disclosed in International (PCT) Patent Application Publication No. WO2015/160911 (International (PCT) Patent Application Serial No. PCT/US2015/025909 as filed on Apr. 15, 2015), is hereby incorporated herein by reference in its entirety for all purposes. Likewise, any ammonia oxidizing bacteria disclosed in International (PCT) Patent Application Publication No. WO2015/160911 (International (PCT) Patent Application Serial No. PCT/US2015/025909 as filed on Apr. 15, 2015), is also hereby incorporated herein by reference in its entirety for all purposes. In certain embodiments, the ammonia oxidizing microorganism is a strain as described therein.

In accordance with one or more embodiments, ammonia oxidizing microorganisms may exist in several metabolic states, e.g. growth state, storage state, and/or polyphosphate loading state.

In accordance with one or more embodiments, ammonia oxidizing microorganisms may have desirable properties, e.g., optimized properties, such as the ability to suppress growth of pathogenic bacteria, and an enhanced ability to produce nitric oxide and nitric oxide precursors.

Optimized Nitrosomonas eutropha (N. eutropha), as that term is used herein, refers to an N. eutropha having an optimized growth rate; an optimized NH₄⁺ oxidation rate; and/or optimized resistance to NH₄⁺. In an embodiment it differs from naturally occurring N. eutropha by at least one nucleotide, e.g., a nucleotide in a gene selected from ammonia monooxygenase, hydroxylamine oxidoreductase, cytochrome c554, and cytochrome c_{M}55². The difference can arise, e.g., through selection of spontaneously arising mutation, induced mutation, or directed genetic engineering, of the N. eutropha. In an embodiment it differs from a naturally occurring N. eutropha in that it has a constellation of alleles, not present together in nature. These differences may provide for one or more of a treatment or prevention of a disease or condition, such as but not limited to one associated with low nitrite levels.

Any ammonia oxidizing bacteria, e.g., N. eutropha, for example N. eutropha referred to as “D23”, also known as “B244” or “AOB D23-100” may have several of the above-described properties. Any ammonia oxidizing archaea (AOA) may also have several of the above-described properties.

The AOBs contemplated in this disclosure may comprise mutations relative to wild-type AOBs. These mutations may, e.g., occur spontaneously, be introduced by random mutagenesis, or be introduced by targeted mutagenesis. For instance, the AOBs may lack one or more genes or regulatory DNA sequences that wild-type AOBs typically comprise. The AOBs may also comprise point mutations, substitutions, insertions, deletions, and/or rearrangements relative to the sequenced strain or a wild-type strain. The AOBs may be a purified preparation of optimized AOBs.

In certain embodiments, the AOBs are transgenic. For instance, it may comprise one or more genes or regulatory DNA sequences that wild-type ammonia oxidizing bacteria lacks. More particularly, the ammonia oxidizing bacteria may comprise, for instance, a reporter gene, a selective marker, a gene encoding an enzyme, or a promoter (including an inducible or repressible promoter). In some embodiments the additional gene or regulatory DNA sequence is integrated into the bacterial chromosome; in some embodiments the additional gene or regulatory DNA sequence is situated on a plasmid.

In some embodiments, the AOBs differ by at least one nucleotide from naturally occurring bacteria. For instance, the AOBs may differ from naturally occurring bacteria in a gene or protein that is part of a relevant pathway, e.g., an ammonia metabolism pathway, a urea metabolism pathway, or a pathway for producing nitric oxide or nitric oxide precursors. More particularly, the AOBs may comprise a mutation that elevates activity of the pathway, e.g., by increasing levels or activity of an element of that pathway.

The above-mentioned mutations can be introduced using any suitable technique. Numerous methods are known for introducing mutations into a given position. For instance, one could use site-directed mutagenesis, oligonucleotide-directed mutagenesis, or site-specific mutagenesis. Non-limiting examples of specific mutagenesis protocols are described in, e.g., Mutagenesis, pp. 13.1-13.105 (Sambrook and Russell, eds., Molecular Cloning A Laboratory Manual, Vol. 3, 3.sup.rd ed. 2001). In addition, non-limiting examples of well-characterized mutagenesis protocols available from commercial vendors include, without limitation, Altered Sites® II in vitro Mutagenesis Systems (Promega Corp., Madison, Wis.); Erase-a-Base® System (Promega, Madison, Wis.); GeneTailor™ Site-Directed Mutagenesis System (Invitrogen, Inc., Carlsbad, Calif.); QuikChange® II Site-Directed Mutagenesis Kits (Stratagene, La Jolla, Calif.); and Transformer™ Site-Directed Mutagenesis Kit (BD-Clontech, Mountain View, Calif.).

In certain embodiments of the disclosure, the ammonia oxidizing microorganisms may be axenic. The preparation (formulation or composition) of ammonia oxidizing microorganisms may comprise, consist essentially of, or consist of axenic ammonia oxidizing microorganisms.

The ammonia oxidizing bacteria of this disclosure may be from a genus selected from the group consisting of Nitrosomonas, Nitrosococcus, Nitrosospria, Nitrosocystis, Nitrosolobus, Nitrosovibrio, and combinations thereof.

This disclosure provides, inter alia, N. eutropha strain D23, a unique, e.g., optimized strain of ammonia oxidizing bacteria that can increase production of nitric oxide and nitric oxide precursors on a surface of a subject, e.g., a human subject. This disclosure also provides methods of administering and using the bacteria and preparations, compositions, formulations, and products, comprising the bacteria.

In embodiments, the ammonia oxidizing bacteria, e.g., N. eutropha is non-naturally occurring. For instance, it may have accumulated desirable mutations during a period of selection. In other embodiments, desirable mutations may be introduced by an experimenter. In some embodiments, the N. eutropha may be a purified preparation, and may be an optimized N. eutropha.

In preferred embodiments, the N. eutropha strain is autotrophic and so incapable of causing infection. A preferred strain utilizes urea as well as ammonia, so that hydrolysis of the urea in sweat would not be necessary prior to absorption and utilization by the bacteria. Also, in order to grow at low pH, the bacteria may either absorb NH₄⁺ ions or urea. The selected strain should also be capable of living on the external skin of a subject, e.g., a human, and be tolerant of conditions there.

Although this disclosure refers to N. eutropha strain D23 in detail, the preparations, methods, compositions, treatments, formulas and products may be used with one or more of: one or more other strains of N. eutropha, one or more other species of Nitrosomonas, and one or more other ammonia oxidizing microorganism, e.g. ammonia oxidizing bacteria or other ammonia oxidizing archaea.

In certain embodiments, a bacterium with the above-mentioned sequence characteristics has one or more of (1) an optimized growth rate as measured by doubling time, (2) an optimized growth rate as measured by OD600, (3) an optimized NH₄⁺ oxidation rate, (4) an optimized resistance to NH₄⁺, and (4) an optimized resistance to NO₂⁻. Particular nonlimiting sub-combinations of these properties are specified in the following paragraph.

In some embodiments, the ammonia oxidizing bacteria, e.g., the N. eutropha described herein, or an axenic composition thereof, has one or more of: (1) an optimized growth rate as measured by doubling time, (2) an optimized growth rate as measured by OD600, (3) an optimized NH₄⁺ oxidation rate, (4) an optimized resistance to, NH₄⁺, and (4) an optimized resistance to, NO₂⁻. For instance, the bacterium may have properties (1) and (2); (2) and (3); (3) and (4); or (4) and (5) from the list at the beginning of this paragraph. As another example, the bacterium may have properties (1), (2), and (3); (1), (2), and (4); (1), (2), and (5); (1), (3), and (4); (1), (3), and (5); (1), (4), and (5); (2), (3), and (4); (2), (3), and (5), or (3), (4), and (5) from the list at the beginning of this paragraph. As a further example, the bacterium may have properties (1), (2), (3), and (4); (1), (2), (3), and (5); (1), (2), (4), and (5); (1), (3), (4), and (5); or (2), (3), (4), and (5) from the list at the beginning of this paragraph. In some embodiments, the bacterium has properties (1), (2), (3), (4), and (5) from the list at the beginning of this paragraph.

In certain embodiments, the N. eutropha strain comprises a nucleic acid sequence, e.g., a genome, that hybridizes to SEQ ID NO: 1 of International (PCT) Patent Application Publication No. WO2015160911 (International (PCT) Patent Application Serial No. PCT/US2015/025909 filed on Apr. 15, 2015), or to the genome of the D23 strain deposited in the form of 25 vials with the ATCC patent depository on Apr. 8, 2014, designated AOB D23-100, under accession number PTA-121157, or their complements, under low stringency, medium stringency, high stringency, or very high stringency, or other hybridization condition.

The D23 strain is not believed to be a product of nature, but rather has acquired certain mutations and characteristics during an extended period of culture and selection in the laboratory. For instance, D23 has an ability to grow in conditions of greater than about 200 or 250 mM NH₄⁺ for more than 24 hours.

In some embodiments, the N. eutropha disclosed herein differ from naturally occurring bacteria in the abundance of siderophores. For instance, the N. eutropha may have elevated or reduced levels of siderophores compared to N. eutropha C91. Generally, siderophores are secreted iron-chelating compounds that help bacteria scavenge iron from their environment. Some siderophores are peptides, and others are small organic molecules.

The practice of the present invention may employ, unless otherwise indicated, conventional methods of immunology, molecular biology, and recombinant DNA techniques within the skill of the art. Such techniques are explained fully in the literature. See, e.g., Sambrook, et al. Molecular Cloning: A Laboratory Manual (Current Edition); and Current Protocols in Molecular Biology (F. M. Ausubel, et al. eds., current edition).

### Select Definitions

An ammonia oxidizing microorganism, e.g., ammonia oxidizing bacteria, refers to a microorganism capable of oxidizing ammonia or ammonium to nitrite at a rate, e.g., a substantial rate, e.g., a pre-determined rate. The rate, e.g., a pre-determined rate, may refer to the conversion of ammonium ions (NH₄⁺) (e.g., at about 200 mM) to nitrite (NO₂⁻), for example, as determined or measured in an in vitro assay or when administered to a subject, e.g., a human. The rate may be a conversion at a rate of at least about 1 picomole per minute per mg protein, 0.01, 0.1, 1, 10, 25, 50, 75, 125, or 150 nanomoles NO₂⁻ per minute per mg protein, e.g., about 0.01-1, 0.1-50, 50-100, 100-150, 75-175, 75-125, 100-125, 125-150, or 125-175 nanomoles/minute/mg protein, e.g., about 125 nanomoles NO₂⁻ per minute per mg protein for a continuous culture, for example having an OD of about 0.5. The rate of conversion may be between about 1 picomole per minute per mg protein to about 1 millimole per minute per mg protein. The rate of conversion may be at most about 1 mole NO₂⁻ per minute per mg protein, e.g. at least about, about, or at most about 1 decimole, 1 centimole, 1 millimole, or 1 micromole NO₂⁻ per minute per mg protein.

As used herein, “axenic” refers to a composition comprising an organism that is substantially free of other organisms. For example, an axenic culture of ammonia oxidizing bacteria is a culture that is substantially free of organisms other than ammonia oxidizing bacteria. For example, an axenic culture of N. eutropha is a culture that is substantially free of organisms other than N. eutropha. In some embodiments, “substantially free” denotes undetectable by a method used to detect other organisms, e.g., plating the culture and examining colony morphology, or PCR for a conserved gene such as 16S RNA. An axenic composition may comprise elements that are not organisms, e.g., it may comprise nutrients or excipients. Any embodiment, preparation, composition, or formulation of ammonia oxidizing bacteria discussed herein may comprise, consist essentially of, or consist of optionally axenic ammonia oxidizing bacteria.

Throughout this disclosure, formulation may refer to a composition or preparation or product.

As used herein, an “autotroph”, e.g., an autotrophic bacterium, is any organism capable of self-nourishment by using inorganic materials as a source of nutrients and using photosynthesis or chemosynthesis as a source of energy. Autotrophic bacteria may synthesize organic compounds from carbon dioxide and ATP derived from other sources, oxidation of ammonia to nitrite, oxidation of hydrogen sulfide, and oxidation of Fe²⁺, to Fe³⁺. Autotrophic bacteria of the present disclosure are incapable of causing infection.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concomitant” or “concurrent delivery”. In other embodiments, the delivery of one treatment ends before the delivery of the other treatment begins. This is sometimes referred to herein as “successive” or “sequential delivery.” In embodiments of either case, the treatment is more effective because of combined administration. For example, the second treatment is a more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive (i.e., synergistic). The delivery can be such that an effect of the first treatment delivered is still detectable when the second is delivered. In some embodiments, one or more treatment may be delivered prior to diagnosis of the patient with the disorder.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “optimized growth rate” refers to one or more of: a doubling time of less than about 4, 5, 6, 7, 8, 9, or 10 hours when cultured under batch conditions as described herein in Example 2; a doubling time of less than about 16, 18, 20, 22, 24, or 26 hours, when grown under chemostat conditions as described herein in Example 2; or growing from an OD600 of about 0.15 to at least about 0.3, 0.4, 0.5, 0.6, 0.7, or 0.8 over about 1 or 2 days. In an embodiment, optimized growth rate is one having a doubling time that it is at least 10, 20, 30, 40, or 50% shorter than that of a naturally occurring N. eutropha.

As used herein, “optimized NH₄⁺ oxidation rate” refers to a rate of at least about 50, 75, 125, or 150 micromoles per minute of converting NH₃ or NH₄⁺ into NO₂⁻. For instance, the rate may be at least about 50, 75, 125, or 150 micromoles per minute of converting NH₄⁺ (e.g., at about 200 mM) to NO₂⁻. In an embodiment, an optimized NH₄⁺ oxidation rate is one in which NH₃ or NH₄⁺ is converted into NO₂⁻ at least 10, 20, 30, 40, or 50% more rapidly than is seen with a naturally occurring N. eutropha.

As used herein, “optimized resistance to NH₄⁺” refers to an ability to grow in conditions of greater than 50, 75, 100, 125, 150, 175, 200, 225, 250, 275, or 300 mM NH₃ or NH₄⁺ for at least about 24 or 48 hours. In an embodiment, an optimized resistance to NH₄⁺ refers to the ability to grow at least 10, 20, 30, 40, or 50% more rapidly, or at least 10, 20, 30, 40, or 50% longer, in the presence of a selected concentration of NH₃ or NH₄⁺ than can a naturally occurring N. eutropha.

As used herein, “transgenic” means comprising one or more exogenous portions of DNA. The exogenous DNA is derived from another organism, e.g., another bacterium, a bacteriophage, an animal, or a plant.

As used herein, treatment of a disease or condition refers to reducing the severity or frequency of at least one symptom of that disease or condition, compared to a similar but untreated patient. Treatment can also refer to halting, slowing, or reversing the progression of a disease or condition, compared to a similar but untreated patient. Treatment may comprise addressing the root cause of the disease and/or one or more symptoms.

As used herein a therapeutically effective amount refers to a dose sufficient to prevent advancement, or to cause regression of a disease or condition, or which is capable of relieving a symptom of a disease or condition, or which is capable of achieving a desired result. A therapeutically effective dose can be measured, for example, as a number of bacteria or number of viable bacteria (e.g., in CFUs) or a mass of bacteria (e.g., in milligrams, grams, or kilograms), or a volume of bacteria (e.g., in mm³).

As used herein, the term “viability” refers to the autotrophic bacteria's, e.g., ammonia oxidizing bacteria's, ability to oxidize ammonia, ammonium, or urea to nitrite at a pre-determined rate. In some embodiments, the rate refers to the conversion of ammonium ions (NH₄⁺) (e.g., at about 200 mM) to nitrite (NO₂⁻) at a rate of at least about 1 picomole, 0.01, 0.1, 1, 10, 25, 50, 75, 125, or 150 nanomoles NO₂⁻ per minute, e.g., about 0.01-1, 0.1-50, 50-100, 100-150, 75-175, 75-125, 100-125, 125-150, or 125-175 nanomoles/minute, e.g., about 125 nanomoles NO₂⁻ per minute. The rate of conversion may be at most about 1 mole NO: per minute, e.g. at least about, about, or at most about 1 decimole, 1 centimole, 1 millimole, or 1 micromole NO₂⁻ per minute. Viable ammonia oxidizing microorganisms may generally comprise culturable AOMs or AOMs that are otherwise able to generate NO, nitrate, or nitrite.

As used herein, a “subject” may include an animal, a mammal, a human, a non-human animal, a livestock animal, or a companion animal. The term “subject” is intended to include human and non-human animals, for example, vertebrates, large animals, and primates. In certain embodiments, the subject is a mammalian subject, and in particular embodiments, the subject is a human subject. Although applications with humans are clearly foreseen, veterinary applications, for example, with non-human animals, are also envisaged herein. The term “non-human animals” of the disclosure includes all vertebrates, for example, non-mammals (such as birds, for example, chickens; amphibians; reptiles) and mammals, such as non-human primates, domesticated, and agriculturally useful animals, for example, sheep, dog, cat, cow, pig, rat, among others.

“Microbiome” refers to a population, e.g, one or more microorganisms that live on a surface of a subject, e.g., in the gut, mouth, skin, and/or elsewhere in a subject. The population may have one or more beneficial functions and/or benefits, relevant to supporting the life of a subject.

“Biome-friendly” refers to something, e.g, a product, e.g., a cosmetic product, e.g., a finished cosmetic product that may allow for minimal disruption of a microbiome of a subject. For example, biome-friendly refers to a product that may be applied to a subject that may allow the microbiome at the point of application to be maintained, minimally disrupted, and/or able to return to the microbiome after a period of time after application of the product. In embodiments, biome-friendly may refer to ammonia oxidizing microorganism-friendly, e.g. ammonia oxidizing bacteria-friendly in that the product may allow for minimal disruption of the ammonia oxidizing bacteria of a subject. In embodiments, “biome-friendly” may be referred to as “biome-compatible.”

A “natural product” is or may comprise a product that may be at least partially derived from nature. It may be anything or comprise anything produced by a living organism, and may include organisms themselves. Natural products may include or comprise an entire organism, and part of an organism (e.g., a leaf of a plant), an extract from an organism, an organic compound from an organism, a purified organic compound from an organism. Natural products may be or comprise organic substances found and cells, including primary metabolites (amino acids, carbohydrates, and nucleic acids) and secondary metabolites (organic compounds found in a limited range of species, e.g., polyketides, fatty acids, terpenoids, steroids, phenylpropanoids, alkaloids, specialized amino acids and peptides, specialized carbohydrates). Natural products may be or comprise polymeric organic materials such as cellulose, lignin, and proteins.

As used herein, “presence” or “level” may refer to a qualitative or quantitative amount of a component, e.g., any one or more of an ammonia oxidizing microorganisms, ammonia, ammonium ions, urea, nitrite, or nitric oxide. The presence or level may include a zero value or a lack of presence of a component.

As used herein, the term “surfactant”, includes compounds that may lower the surface tension, or interfacial tension, between two liquids or between a liquid and a solid. Surfactants may act as detergents, wetting agents, emulsifiers, foaming agents, and dispersants. Surfactants may include one or more of the following, alone, or in combination with those listed, or other surfactants or surfactant-like compounds: cocamidopropyl betaine (ColaTeric COAB), polyethylene sorbitol ester (e.g., Tween 80), ethoxylated lauryl alcohol (RhodaSurf 6 NAT), sodium laureth sulfate/lauryl glucoside/cocamidopropyl betaine (Plantapon 611 L UP), sodium laureth sulfate (e.g., RhodaPex ESB 70 NAT), alkyl polyglucoside (e.g., Plantaren 2000 N UP), sodium laureth sulfate (Plantaren 200), Dr. Bronner's Castile soap, Dr. Bronner's baby soap, Lauramine oxide (ColaLux Lo), sodium dodecyl sulfate (SDS), polysulfonate alkyl polyglucoside (PolySufanate 160 P), sodium lauryl sulfate (Stepanol-WA Extra K), and combinations thereof. Dr. Bronner's Castile soap and baby soap comprises water, organic coconut oil, potassium hydroxide, organic olive oil, organic fair deal hemp oil, organic jojoba oil, citric acid, and tocopherol. Surfactants may include Sodium Laurylglucosides Hydroxypropylsulfonate (Suga® nate 160NC), lauramidopropyl betaine (Cola® Teric LMB); Cocamidopropyl hydroxysultaine (Cola® Teric CBS); disodium cocoamphodiacetate (Cola® Teric CDCX-LV); sodium laurylglucosides hydroxypropyl phosphate (Suga® Fax D12). Surfactants may include sodium lauroyl methyl isethionate (Iselux® LQ-CLR-SB); sodium methyl cocoyl taurate (Pureact WS Conc.); Aqua (and) Sodium Lauroyl Methyl Isethionate (and) Cocamidopropyl Betaine (and) Sodium Cocoyl Isethionate (and) Sodium Methyl Oleoyl Taurate (Iselux® SFS-SB). Other surfactants are contemplated by this disclosure.

### Preparations, Compositions, Formulations, and Products Comprising Ammonia Oxidizing Microorganisms

The present disclosure provides, inter alia, compositions comprising ammonia oxidizing microorganisms, preparations, e.g., purified and/or optimized preparations, comprising AOM, formulations comprising AOM, and various products comprising AOM, e.g., a natural product, a non-natural product, a fortified natural product, a consumer product, a therapeutic product, or a cosmetic product. The terms preparation, composition, formulation, and product may be used interchangeably herein.

Any embodiment, preparation, composition, formulation, or product of ammonia oxidizing microorganisms discussed herein may comprise, consist essentially of, or consist of (optionally axenic) ammonia oxidizing microorganisms, e.g., live ammonia oxidizing microorganisms.

The preparation may comprise or be supplemented with a product or byproduct of an ammonia oxidizing microorganism, e.g., nitrite, nitrate, nitric oxide, CoQ8. In at least some embodiments, the preparation may comprise or be supplemented with a composition that promotes growth or metabolism of ammonia oxidizing microorganisms, promotes production of products or byproducts of ammonia oxidizing microorganisms, promotes urease activity, or has a synergistic effect with ammonia oxidizing microorganisms, e.g., ammonia, ammonium salts, urea, and urease. For instance, the preparation may be supplemented with one or more of NO, nitrite, nitrate, CoQ8, ammonia, ammonium salts, urea, and urease. The supplement may be comprised in the same formulation as the ammonia oxidizing microorganisms or in a separate formulation for concurrent or combination administration. The supplement formulation may be prepared for delivery via any delivery mode, for example inhaled forms of NO, nitrite, or nitrate. The preparation may comprise, inter alia, at least one of ammonia, ammonium salts, and urea. The preparation may comprise or be supplemented with an anti-inflammatory agent or a composition that provides an anti-inflammatory effect.

The present disclosure provides for preparations comprising ammonia oxidizing microorganisms for cosmetic use.

The present disclosure provides for preparations comprising ammonia oxidizing microorganisms for therapeutic use.

In some embodiments, a preparation of ammonia oxidizing microorganisms may comprise a concentration or amount, e.g., an effective amount, of ammonia oxidizing microorganisms sufficient to have a desired cosmetic effect. The preparation may be formulated and/or delivered to impart the desired cosmetic effect locally and/or systemically.

In some embodiments, a preparation of ammonia oxidizing microorganisms may comprise a concentration or amount, e.g., an effective amount, of ammonia oxidizing microorganisms sufficient to have a desired therapeutic effect, e.g., to at least partially treat a condition or disease. The preparation may be formulated and/or delivered to impart the desired therapeutic effect locally and/or systemically.

In some embodiments, a preparation of ammonia oxidizing microorganisms may comprise a concentration or amount, e.g., an effective amount, of ammonia oxidizing microorganisms sufficient to alter, e.g., reduce or increase, an amount, concentration or proportion of a bacterium, or genus of bacteria in a subject. The bacteria may be non-pathogenic or pathogenic, or potentially pathogenic.

In some embodiments, a preparation of ammonia oxidizing microorganisms may comprise a concentration or amount, e.g., an effective amount, of ammonia oxidizing microorganisms sufficient to modulate a microbiome associated with a subject.

In some embodiments, a preparation of ammonia oxidizing microorganisms may comprise a concentration or amount, e.g., an effective amount, of ammonia oxidizing microorganisms sufficient to deliver NO to a subject. A preparation of ammonia oxidizing microorganisms may comprise a concentration or amount, e.g., an effective amount, of ammonia oxidizing microorganisms such that when administered, the preparation modulates, changes, or alters a level of nitrite or NO at a target tissue or in circulation. For instance, a preparation of ammonia oxidizing microorganisms may comprise a concentration or amount, e.g., an effective amount, of ammonia oxidizing microorganisms such that when administered, the preparation results in an increased level of nitrite or NO at a target tissue or in circulation.

The present disclosure provides, inter alia, non-limiting compositions comprising ammonia oxidizing microorganisms, e.g., N. eutropha, e.g., a purified preparation of an optimized N. eutropha. In some embodiments, the N. eutropha in the compositions has at least one property selected from an optimized growth rate, an optimized NH₄⁺ oxidation rate, and an optimized resistance to NH₄⁺.

In some aspects, the present disclosure provides compositions with a defined number of species. A composition may include only one type of species, e.g., one type of ammonia oxidizing microorganism. This disclosure also provides a composition having, e.g., N. eutropha and one other type of organism, and no other types of organism. In other examples, the composition has, e.g., N. eutropha and 2, 3, 4, 5, 6, 7, 8, 9, or 10 other types of organism, and no other types of organism. The other type of organism in this composition may be, for instance, a bacterium, such as an ammonia-oxidizing bacterium. Suitable ammonia-oxidizing microorganisms for this purpose include those in the genera Nitrosomonas, Nitrosococcus, Nitrosospira, Nitrosocystis, Nitrosolobus, or Nitrosovibrio. Likewise, the composition may also include AOA.

In some embodiments, the composition comprising, e.g., N. eutropha provides conditions that support N. eutropha viability. For instance, the composition may promote N. eutropha growth and metabolism or may promote a dormant state (e.g., freezing) from which viable N. eutropha can be recovered. When the composition promotes growth or metabolism, it may contain water and/or nutrients that N. eutropha consumes, e.g., as ammonium, ammonia, urea, oxygen, carbon dioxide, or trace minerals. In some embodiments, the composition comprising ammonia oxidizing microorganisms provides conditions that support ammonia oxidizing microorganisms viability. For instance, the composition may promote ammonia oxidizing microorganisms growth and metabolism or may promote a dormant state (e.g., freezing) or storage state as described herein, from which viable ammonia oxidizing microorganisms can be recovered. When the composition promotes growth or metabolism, it may contain water and/or nutrients that ammonia oxidizing microorganisms consumes, e.g., as ammonium ions, ammonia, urea, oxygen, carbon dioxide, or trace minerals.

In some embodiments, one or more other organisms, for example, organisms besides ammonia oxidizing microorganisms, may be included in the preparation of ammonia oxidizing microorganisms. For example, a community of organisms or an organism of the genus selected from the group consisting of Lactobacillus, Streptococcus, Bifidobacter, and combinations thereof, may be provided in the preparation of ammonia oxidizing microorganisms. In some embodiments, the preparation may be substantially free of other organisms.

Preparations of ammonia oxidizing microorganisms may comprise between about between about 10³ to about 10¹⁴ CFU/ml. In some embodiments, the preparation of ammonia oxidizing microorganisms may comprise at least about or greater than about 10³, 10⁴, 10⁵, 10⁶, 10⁷, 10⁸, 10⁹, 10¹⁰, 10¹¹, 2×10¹¹, 5×10¹¹, 10¹², 2×10¹², 5×10¹², 10¹³, 2×10¹³, 5×10¹³, or 10¹⁴; or about 10³-10⁴, 10⁴-10⁵, 10⁶-10⁷, 10⁷-10⁸, 10⁸-10⁹, 10⁹-10¹⁰, 10¹⁰-10¹¹, 10¹¹-10¹², 10¹²-10¹³, or 10¹³-10¹⁴ CFU/ml.

In some embodiments, a preparation of ammonia oxidizing microorganisms may comprise between about 1×10⁹ to about 10×10⁹ CFU/ml. In some embodiments, an administered dose of the preparation may comprise about 3×10¹⁰ CFU, e.g., 3×10¹⁰ CFU per day. In some embodiments, an administered dose of the preparation may comprise about 1×10⁹ to about 10×10⁹ CFU per day, e.g., about 1×10⁹ to about 10×10⁹ CFU per day. In some embodiments, an administered dose of the preparation may comprise about 10³, 10⁴, 10⁵, 10⁶, 10⁷, 10⁸, 10⁹, 10¹⁰, 10¹¹, 2×10¹¹, 5×10¹¹, 10¹², 2×10¹², 5×10¹², 10¹³, 2×10¹³, 5×10¹³, or 10¹⁴; or about 10³-10⁴, 10⁴-10⁵, 10⁶-10⁷, 10⁷-10⁸, 10⁸-10⁹, 10⁹-10¹⁰, 10¹⁰-10¹¹, 10¹¹-10¹², 10¹²-10¹³, or 10¹³-10¹⁴ CFU per administration or per day.

In some embodiments, an administered dose of the preparation may comprise at least about 7×10¹⁰ CFU, e.g., 21×10¹⁰ CFU per week. In some embodiments, an administered dose of the preparation may comprise about 1×10⁹ to about 10×10⁹ CFU per week, e.g., about 1×10⁹ to about 10×10⁹ CFU per week. In some embodiments, an administered dose of the preparation may comprise about or greater than about 10³, 10⁴, 10⁵, 10⁶, 10⁷, 10⁸, 10⁹, 10¹⁰, 10¹¹, 2×10¹¹, 5×10¹¹, 10¹², 2×10¹², 5×10¹², 10¹³, 2×10¹³, 5×10¹³, or 10¹⁴; or about 10³-10⁴, 10⁴-10⁵, 10⁶-10⁷, 10⁷-10⁸, 10⁸-10⁹, 10⁹-10¹⁰, 10¹⁰-10¹¹, 10¹¹-10¹², 10¹²-10¹³, or 10¹³-10¹⁴ CFU per week.

In some embodiments, an administered dose of the preparation may comprise at least about 30×10¹⁰ CFU, e.g., 90×10¹⁰ CFU per month. In some embodiments, an administered dose of the preparation may comprise about 1×10⁹ to about 10×10⁹ CFU per month, e.g., about 1×10⁹ to about 10×10⁹ CFU per month. In some embodiments, an administered dose of the preparation may comprise about or greater than about 10³, 10⁴, 10⁵, 10⁶, 10⁷, 10⁸, 10⁹, 10¹⁰, 10¹¹, 2×10¹¹, 5×10¹¹, 10¹², 2×10¹², 5×10¹², 10¹³, 2×10¹³, 5×10¹³, or 10¹⁴; or about 10³-10⁴, 10⁴-10⁵, 10⁶-10⁷, 10⁷-10⁸, 10⁸-10⁹, 10⁹-10¹⁰, 10¹⁰-10¹¹, 10¹¹-10¹², 10¹²-10¹³, or 10¹³-10¹⁴ CFU per month.

In some embodiments, the preparation of ammonia oxidizing microorganisms may comprise between about 0.1 milligrams (mg) and about 1000 mg of ammonia oxidizing microorganisms. In certain aspects, the preparation may comprise between about 50 mg and about 1000 mg of ammonia oxidizing microorganisms. The preparation may comprise between about 0.1-0.5 mg, 0.2-0.7 mg, 0.5-1.0 mg, 0.5-2 mg, 0.5-5 mg, 2.5-5 mg, 2.5-7.0 mg, 5.0-10 mg, 7.5-15 mg, 10-15 mg, 15-20 mg, 15-25 mg, 20-30 mg, 25-50 mg, 25-75 mg, 50-75 mg, 50-100 mg, 75-100 mg, 100-200 mg, 200-300 mg, 300-400 mg, 400-500 mg, 500-600 mg, 600-700 mg, 700-800 mg, 800-900 mg, 900-1000 mg, 100-250 mg, 250-500 mg, 100-500 mg, 500-750 mg, 750-1000 mg, or 500-1000 mg.

Advantageously, a formulation may have a pH level that promotes AOM, e.g., N. eutropha viability, e.g., metabolic activity. Urea would hydrolyze to ammonia and would raise the pH to 7 to 8. AOB are very active at this pH range and would lower the pH to about 6 where the NH₃ converts to ammonium and is unavailable. Lower pH levels, e.g. about pH 4, are also acceptable.

The ammonia oxidizing microorganisms, e.g., N. eutropha may be combined with one or more pharmaceutically or cosmetically acceptable excipients. In some embodiments, “pharmaceutically acceptable excipient” refers to a pharmaceutically-acceptable material, composition, or vehicle, such as a liquid or solid filler, diluent, solvent, or encapsulating material. In some embodiments, each excipient is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

In some embodiments, a cosmetically acceptable excipient refers to a cosmetically acceptable material, composition, or vehicle, such as a liquid or solid filler, diluent, solvent, or encapsulating material. In some embodiments, each excipient is cosmetically acceptable in the sense of being compatible with the other ingredients of a cosmetic formulation, and suitable for use in contact with the tissue or organ of humans and animals without excessive toxicity, irritation, allergic response, immunogenicity, or other problems or complications, commensurate with a reasonable benefit/risk ratio.

While it is possible for the active ingredient, e.g., ammonia oxidizing microorganisms, e.g., N. eutropha, to be administered alone, in many embodiments it is present in a pharmaceutical formulation or composition. Accordingly, this disclosure provides a pharmaceutical formulation comprising ammonia oxidizing microorganisms, for example, N. eutropha and a pharmaceutically acceptable excipient. Pharmaceutical compositions may take the form of a pharmaceutical formulation as described below.

In accordance with one or more embodiments, a preparation of ammonia oxidizing microorganisms may be formulated in order to facilitate a desired delivery mechanism or mode of administration thereof. The formulations, e.g., pharmaceutical or cosmetic formulations, described herein include those suitable for, e.g., oral, enteral (including buccal, sublingual, sublabial, and rectal), parenteral (including subcutaneous, intradermal, intramuscular, intravenous, and intraarticular), inhalation (including fine particle dusts or mists which may be generated by means of various types of metered doses, pressurized aerosols, nebulizers or insufflators, and including intranasally or via the lungs), intranasal, eye, ear, rectal, injection, urogenital, and topical (including dermal, transdermal, transmucosal, buccal, sublingual, and intraocular) administration, although the most suitable route may depend upon, for example, a condition or disorder of a recipient.

In accordance with one or more non-limiting embodiments, a preparation comprising ammonia oxidizing microorganisms may be administered to a subject, e.g., for cosmetic or therapeutic purposes, as a solution, suspension, powder, liquid, drop, spray, aerosol, mist, emulsion, foam, cream, ointment, gel, hydrogel, resin, tablet, capsule, film, suppository, enema, douche, pessary, insert, patch, e.g., transdermal patch, or implantable device, e.g., stent, catheter, vaginal ring, or intrauterine device.

Devices configured to deliver a preparation comprising live ammonia oxidizing microorganisms via a desired mode of administration or otherwise via targeted delivery are also disclosed.

In accordance with one or more embodiments, the preparation may be formulated for targeted delivery to a subject, e.g., to a target tissue, region, system, or organ of a subject. For example, the preparation may be formulated for delivery to the eye, ear, nose, urogenital system, respiratory system, or gastrointestinal system of the subject. In some embodiments, targeted delivery may be based on a condition or disorder of a subject. For instance, formulation for targeted delivery may be based on a desired local or systemic effect to be achieved, e.g., a local or systemic therapeutic or cosmetic effect. In some embodiments, a target tissue, region, system, or organ of a subject may be selected for its association with a desired local or systemic effect.

The formulations may conveniently be presented in unit dosage form and may be prepared by any of the methods known in the art of pharmacy. Typically, methods include the step of bringing the active ingredient (e.g., ammonia oxidizing microorganisms, e.g., N. eutropha) into association with a pharmaceutical carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Formulations may be presented as discrete units such as capsules, cachets or tablets, each containing a predetermined amount of, e.g., N. eutropha; as a powder or granules; as a solution or a suspension in an aqueous liquid or a non-aqueous liquid; or as an oil-in-water liquid emulsion or a water-in-oil liquid emulsion. Formulations, e.g., solutions, aerosols, sprays, and mists, may be presented in multi-dosage form, e.g., packaged units including a predetermined number of dosages, or single dosage form, e.g., packaged units including a single dose. The active ingredient may also be presented as a bolus, electuary or paste. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2 S, 1988.

The ammonia oxidizing microorganisms, e.g., N. eutropha compositions can, for example, be administered in a form suitable for immediate release or extended release. Suitable examples of sustained-release systems include suitable polymeric materials, for example semi-permeable polymer matrices in the form of shaped articles, e.g., films, or microcapsules; suitable hydrophobic materials, for example as an emulsion in an acceptable oil; or ion exchange resins. Sustained-release systems may be administered orally; rectally; parenterally; intracisternally; intravaginally; intraperitoneally; topically, for example as a powder, ointment, gel, drop or transdermal patch; bucally; or as a spray.

Preparations for administration can be suitably formulated to give controlled release of ammonia oxidizing microorganisms, e.g., N. eutropha. For example, the pharmaceutical compositions may be in the form of particles comprising one or more of biodegradable polymers, polysaccharide jellifying and/or bioadhesive polymers, or amphiphilic polymers. These compositions exhibit certain biocompatibility features which allow a controlled release of an active substance. See U.S. Pat. No. 5,700,486.

Exemplary compositions include suspensions which can contain, for example, microcrystalline cellulose for imparting bulk, alginic acid or sodium alginate as a suspending agent, methylcellulose as a viscosity enhancer, dicalcium phosphate, starch, magnesium stearate and/or lactose and/or other excipients, binders, extenders, disintegrants, diluents and lubricants, mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use. The surfactant may be a zwitterionic surfactant, a non-ionic surfactant, or an anionic surfactant.

Excipients, such as surfactants that may be used with embodiments of the present disclosure may include one or more of cocamidopropyl betaine (ColaTeric COAB), polyethylene sorbitol ester (e.g., Tween 80), ethoxylated lauryl alcohol (RhodaSurf 6 NAT), sodium laureth sulfate/lauryl glucoside/cocamidopropyl betaine (Plantapon 611 L UP), sodium laureth sulfate (e.g., RhodaPex ESB 70 NAT), alkyl polyglucoside (e.g., Plantaren 2000 N UP), sodium laureth sulfate (Plantaren 200), Dr. Bronner's Castile soap, Dr. Bronner's Castile baby soap, Lauramine oxide (ColaLux Lo), sodium dodecyl sulfate (SDS), polysulfonate alkyl polyglucoside (PolySufanate 160 P), sodium lauryl sulfate (Stepanol-WA Extra K), and combinations thereof. Dr. Bronner's Castile soap and Dr. Bronner's baby soap comprises water, organic coconut oil, potassium hydroxide, organic olive oil, organic fair deal hemp oil, organic jojoba oil, citric acid, and tocopherol.

In some embodiments, surfactants may be used with ammonia oxidizing microorganisms in amounts that allow nitrite production to occur. In some embodiments, the preparation may have less than about 0.0001% to about 10% of surfactant. In some embodiments, the preparation may have between about 0.1% and about 10% surfactant. In some embodiments, the concentration of surfactant used may be between about 0.0001% and about 10%. In some embodiments, the preparation may be substantially free of surfactant.

In some embodiments, the formulation, e.g., preparation, may include other components that may enhance effectiveness of ammonia oxidizing microorganisms, delivery thereof, or enhance a treatment or indication.

In some embodiments, a chelator may be included in the preparation. A chelator may be a compound that may bind with another compound, e.g., a metal. The chelator may provide assistance in removing an unwanted compound from an environment, or may act in a protective manner to reduce or eliminate contact of a particular compound with an environment, e.g., ammonia oxidizing microorganisms, e.g. a preparation of ammonia oxidizing microorganisms, e.g., an excipient. In some embodiments, the preparation may be substantially free of chelator.

Formulations may also contain anti-oxidants, buffers, bacteriostats that prevent the growth of undesired microorganisms, solutes, and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of a sterile liquid carrier, for example saline or water-for-injection, immediately prior to use. Extemporaneous solutions and suspensions may be prepared from powders, granules and tablets of the kind previously described. Exemplary compositions include solutions or suspensions which can contain, for example, suitable non-toxic, pharmaceutically acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor. An aqueous carrier may be, for example, an isotonic buffer solution at a pH of from about 3.0 to about 8.0, a pH of from about 3.5 to about 7.4, for example from 3.5 to 6.0, for example from 3.5 to about 5.0. Useful buffers include sodium citrate-citric acid and sodium phosphate-phosphoric acid, and sodium acetate/acetic acid buffers. The composition in some embodiments does not include oxidizing agents.

Excipients that can be included are, for instance, proteins, such as human serum albumin or plasma preparations. If desired, the pharmaceutical composition may also contain minor amounts of non-toxic auxiliary substances, such as wetting or emulsifying agents, preservatives, and pH buffering agents and the like, for example sodium acetate or sorbitan monolaurate. In some embodiments, excipients, e.g., a pharmaceutically acceptable excipient or a cosmetically acceptable excipient, may comprise an anti-adherent, binder, coat, disintegrant, filler, flavor, color, lubricant, glidant, sorbent, preservative, or sweetener. In some embodiments, the preparation may be substantially free of excipients.

In some embodiments, the preparation may be substantially free of one or more of the compounds or substances listed in the disclosure.

Exemplary compositions for spray, aerosol, or mist administration include solutions in saline, which can contain, for example, benzyl alcohol or other suitable preservatives, absorption promoters to enhance bioavailability, and/or other solubilizing or dispersing agents. Conveniently in compositions for aerosol administration the ammonia oxidizing microorganisms, e.g., N. eutropha is delivered in the form of an aerosol spray presentation from a pressurized pack or a nebulizer, with the use of a suitable propellant, e.g., dichlorodifluoro-methane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin can be formulated to contain a powder mix of the N. eutropha and a suitable powder base, for example lactose or starch. In certain embodiments, N. eutropha is administered as an aerosol from a metered dose valve, through an aerosol adapter also known as an actuator. Optionally, a stabilizer is also included, and/or porous particles for deep lung delivery are included (e.g., see U.S. Pat. No. 6,447,743).

Formulations may be presented with carriers such as cocoa butter, synthetic glyceride esters or polyethylene glycol. Such carriers are typically solid at ordinary temperatures, but liquefy and/or dissolve at body temperature to release the ammonia oxidizing bacteria, e.g., N. eutropha.

Exemplary compositions for topical administration include a topical carrier such as Plastibase (mineral oil gelled with polyethylene). In some aspects, the composition and/or excipient may be in the form of one or more of a liquid, a solid, or a gel. For example, liquid suspensions may include, but are not limited to, water, saline, phosphate-buffered saline, or an ammonia oxidizing storage buffer. Gel formulations may include, but are not limited to agar, silica, polyacrylic acid (for example Carbopol®), carboxymethyl cellulose, starch, guar gum, alginate or chitosan. In some embodiments, the formulation may be supplemented with an ammonia source including, but not limited to ammonium chloride or ammonium sulfate.

In some embodiments, an ammonia oxidizing microorganism, e.g., N. eutropha composition is formulated to improve NO penetration, e.g., into the skin or other target tissue. A gel-forming material such as KY jelly or various hair gels would present a diffusion barrier to NO loss to ambient air, and so improve the skin's absorption of NO. The NO level in the skin will generally not greatly exceed 20 nM/L because that level activates GC and would cause local vasodilatation and oxidative destruction of excess NO.

It should be understood that in addition to the ingredients particularly mentioned above, the formulations as described herein may include other agents conventional in the art having regard to the type of formulation in question.

The formulation, e.g., preparation, e.g., composition may be provided in a container, delivery system, or delivery device, having a weight, including or not including the contents of the container, that may be less than about 50, 100, 200, 300, 400, 500, 600, 700, 800, 900, 1000, 1500, or 2000 grams.

Suitable unit dosage formulations are those containing an effective dose, as hereinbefore recited, or an appropriate fraction thereof, of ammonia oxidizing microorganisms, e.g., N. eutropha.

A therapeutically effective amount of ammonia oxidizing microorganisms, e.g., N. eutropha may be administered as a single pulse dose, as a bolus dose, or as pulse doses administered over time. Thus, in pulse doses, a bolus administration of ammonia oxidizing microorganisms, e.g., N. eutropha is provided, followed by a time period wherein ammonia oxidizing microorganisms, e.g., N. eutropha is administered to the subject, followed by a second bolus administration. In specific, non-limiting examples, pulse doses are administered during the course of a day, during the course of a week, or during the course of a month.

In some embodiments, a preparation of ammonia oxidizing microorganisms, e.g., a formulation, e.g., a composition, may be applied for a pre-determined number of days. This may be based, for example, at least in part, on the severity of the condition or disease, the response to the treatment, the dosage applied and the frequency of the dose. For example, the preparation may be applied for about 1-3, 3-5, 5-7, 7-9, 5-10, 10-14, 12-18, 12-21, 21-28, 28-35, 35-42, 42-49, 49-56, 46-63, 63-70, 70-77, 77-84, 84-91 days, for about 1 month, for about 2 months, for about 3 months. In some embodiments, the ammonia oxidizing bacteria is administered for an indefinite period of time, e.g., greater than one year, greater than 5 years, greater than 10 years, greater than 15 years, greater than 30 years, greater than 50 years, greater than 75 years. In certain aspects, the preparation may be applied for about 16 days.

In some embodiments, a preparation of ammonia oxidizing microorganisms, e.g., a formulation, e.g., a composition, may be applied a pre-determined number of times per day. This may be based, for example, at least in part, on the severity of the condition or disease, the response to the treatment, the dosage applied and the frequency of the dose. For example, the preparation may be applied 1, 2, 3, 4, 5, 6, 7, 8, 9, 10, 11, 12, 13, 14, 15, 16, 17, 18, 19, 20, 21, 22, 23, 24 times per day.

In some embodiments, the preparation may be applied one time per day. In other embodiments, the preparation may be applied two times per day. In some embodiments, the preparation may be applied a first pre-determined amount for a certain number of days, and a second pre-determined amount for a certain subsequent number of days. In some embodiments, the preparation may be applied for about 16 days.

In accordance with one or more embodiments, the preparation may generally be compatible with a physiological environment associated with the subject. In at least some embodiments, compositions are formulated to have a substantially neutral pH or a physiological pH, for instance a pH that normally prevails in the target site for intended delivery, administration, or desired effect. Compositions may be formulated to have a pH between about 5.5 and about 8.5. Compositions may be formulated to comprise compatible conditions, e.g., pH, tonicity, with the target site of physiological environment associated with the subject.

The preparation may be formulated for transmucosal delivery and/or circulation, e.g. locally or systemically. In some embodiments, the preparation may be formulated such that ammonia oxidizing microorganisms, products thereof, or byproducts thereof (e.g., nitrate, nitrite, NO, or CoQ8) penetrate a deposit or target tissue at least 10%, 20%, 30%, 40%, 50%, 60%, 70%, 80%, 90%, or 100%. The preparation may be formulated such that 10%, 20%, 30%, 40%, 50%, 60%, 70%, 80%, 90%, or 100% of ammonia oxidizing microorganisms, products thereof, or byproducts thereof, penetrate a deposit or target tissue or enter circulation.

In accordance with one or more embodiments, the preparation may be in the form of a solution, suspension, emulsion, cream, ointment, gel, hydrogel, or liquid, e.g. drop, spray, aerosol, or mist, tablet, capsule, or device for administration to a subject.

In accordance with one or more embodiments, a preparation, composition, formulation, or product comprising ammonia oxidizing microorganisms may undergo quality control and/or testing while it is being made and/or upon its completion. International (PCT) Patent Application Publication No. WO2015/179669 (International (PCT) Patent Application Serial No. PCT/US2015/032017 as filed on May 21, 2015) which is hereby incorporated herein by reference in its entirety for all purposes describes various methods of preparing materials with ammonia oxidizing microorganisms and of testing such materials. For example, one or more parameters such as OD level, pH level, waste level, nutrient level, contaminant level, oxidation rate, nitrite level, protein concentration may be compared against a predetermined value to assess or evaluate a preparation comprising ammonia oxidizing microorganisms.

The present disclosure provides, inter alia, a kit comprising preparations of ammonia oxidizing microorganisms, as disclosed herein. Formulations may comprise discrete units, e.g., solid, liquid, or gas formulations of ammonia oxidizing microorganisms. Formulations, e.g., solutions, aerosols, sprays, and mists, may be presented in multi-dosage form (multiple use), e.g., packaged units including a predetermined number of dosages, or single dosage form (single use), e.g., packaged units including a single dose. Preparations of ammonia oxidizing microorganisms may be packaged in devices or containers configured to hold a volume of at least about less than 1 ml, 1 ml, 5 ml, 10 ml, 20 ml, 25 ml, 40 ml, 50 ml, 60 ml, 70 ml, 80 ml, 90 ml, 100 ml, or more than about 100 ml.

Kits may further comprise one or more device for administration of the preparation, for example, syringe, needle, catheter, enema, bulb, pipette (eye or ear dropper), and other devices for drug administration as known in the art. Kits may comprise instructions for use, for example instructions for administration of ammonia oxidizing microorganisms as disclosed herein or instructions for combination therapy including administration of ammonia oxidizing microorganisms. Kits may comprise a second or subsequent composition for administration in conjunction with an ammonia oxidizing preparation, as disclosed herein. For instance, kits may comprise a supplement or composition comprising a product or byproduct of ammonia oxidizing microorganisms, a composition that promotes growth or metabolism of ammonia oxidizing microorganisms, a composition that promotes production of products or byproducts of ammonia oxidizing microorganisms, a composition that promotes urease activity, or a composition that has a synergistic effect with ammonia oxidizing microorganisms, or a composition or pharmaceutical agent that treats, e.g., is approved to treat or commonly used to treat, a relevant disease, disorder, or a symptom of a relevant disease or disorder, for example an anti-inflammatory composition. Kits may comprise “biome-friendly” or “biome-compatible” products as disclosed herein, for example one or more microbiome-compatible cosmetic products. Any of the products contained in the kit may be specifically formulated to treat a target indication and/or formulated for a desired mode of delivery, as described herein.

### Natural Products; Consumer Products

In some specific embodiments, a preparation comprising ammonia oxidizing microorganisms as discussed herein may be a natural product or a consumer product. In other embodiments, a preparation of ammonia oxidizing microorganism may instead be used in conjunction with a natural product or consumer product.

Ammonia oxidizing microorganisms, e.g., N. eutropha may be associated with a variety of natural products, and examples of such products are set out below. These natural products may be comprised of formulations, compositions, or preparations disclosed throughout this disclosure.

Natural products may be or comprise products for commercial purposes, and may refer to cosmetics, dietary supplements, and foods, e.g., food, food supplements, medical food, food additive, nutraceutical, or drink, produced from natural sources. Natural products may have pharmacological or biological activity that may be of therapeutic benefit, e.g., in treating disease or conditions. Natural products may be included in traditional medicines, treatments for cosmetological purposes, and spa treatments. A natural product referred to herein may comprise any one or more of the components described as a natural product to be incorporated into a preparation or formulation comprising one or more other components, e.g., excipients. The preparation or formulation referred to as a natural product may comprise a natural product defined herein and one or more additional components or ingredients. Any of the compositions, preparations, or formulations discussed throughout this disclosure may be or comprise one or more natural products.

In some embodiments, the natural product or the fortified natural product may comprise at least one of mud, water, food-derived products, plant-derived products, extracts, and oils. The natural product or the fortified natural product may be used in a spa treatment. In some embodiments, the natural product or the fortified natural product may be incorporated into at least one of a powder, cream, lotion, wrap, scrub, eye mask, facial mask, body mask, aerosol, e.g., mist, spray, salve, wipe, stick, bandage, or soak.

In some embodiments, the natural product or fortified natural product may be provided as, or may be disposed in at least one of a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

Ammonia oxidizing microorganisms, e.g., N. eutropha may be associated with a variety of consumer products, and examples of such products are set out below and be comprised of formulations, compositions, or preparations disclosed throughout this disclosure. In some embodiments, the ammonia oxidizing bacteria, e.g., N. eutropha associated with a product is admixed with the product, for example, spread evenly throughout the product, and in some embodiments, ammonia oxidizing bacteria, e.g., the N. eutropha associated with a product is layered on the product.

In some embodiments, the preparation may be disposed in, or provided as, a powder, cosmetic, cream, stick, aerosol, e.g., mist, salve, wipe, or bandage.

In some embodiments, ammonia oxidizing bacteria, e.g., N. eutropha is associated with a powder. Powders are typically small particulate solids that are not attached to each other and that can flow freely when tilted. Exemplary powders for consumer use include talcum powder and some cosmetics (e.g., powder foundation).

In some embodiments, the ammonia oxidizing bacteria is associated with a cosmetic. The cosmetic may be a substance for topical application intended to alter a person's appearance, e.g., a liquid foundation, a powder foundation, blush, or lipstick, and may be referred to as a preparation. The cosmetic may be any substance recited in the Food and Drug Administration regulations, e.g., under 21 C.F.R. § 720.4.

In some embodiments, ammonia oxidizing bacteria, e.g., N. eutropha is associated with a cosmetic. The cosmetic may be a substance for topical application intended to alter a person's appearance, e.g., a liquid foundation, a powder foundation, blush, or lipstick. Other components may be added to these cosmetic preparations as selected by one skilled in the art of cosmetic formulation such as, for example, water, mineral oil, coloring agent, perfume, aloe, glycerin, sodium chloride, sodium bicarbonate, pH buffers, UV blocking agents, silicone oil, natural oils, vitamin E, herbal concentrates, lactic acid, citric acid, talc, clay, calcium carbonate, magnesium carbonate, zinc oxide, starch, urea, and erythorbic acid, or any other excipient known by one of skill in the art, including those disclosed herein.

The preparation, e.g., the cosmetic, may be at least one of a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

In some embodiments, the formulations, compositions, or preparations described herein, may comprise, be provided as, or disposed in at least one of a baby product, e.g., a baby shampoo, a baby lotion, a baby oil, a baby powder, a baby cream; a bath preparation, e.g., a bath oil, a tablet, a salt, a bubble bath, a bath capsule; a powder (dusting and talcum), a sachet; hair preparations, e.g., hair conditioners, rinses, shampoos, tonics, face powders, cuticle softeners, nail creams and lotions, oral hygiene products, mouthwashes, bath soaps, douches, feminine hygiene deodorants; shaving preparations, e.g., aftershave lotions, skin care preparations, e.g., cleansing, face and neck, body and hand, foot powders and sprays, moisturizing, night preparations, paste masks, skin fresheners; and suntan preparations, e.g., gels, creams, and liquids.

In some embodiments, ammonia oxidizing microorganisms, e.g., the N. eutropha is associated with an aerosol, spray, or mist and these terms may be used interchangeably. An aerosol is typically a colloid of fine solid particles or fine liquid droplets, in a gas such as air. Aerosols may be created by placing the N. eutropha (and optionally carriers) in a vessel under pressure, and then opening a valve to release the contents. The container may be designed to only exert levels of pressure that are compatible with N. eutropha viability. For instance, the high pressure may be exerted for only a short time, and/or the pressure may be low enough not to impair viability. Examples of consumer uses of aerosols include for sunscreen, deodorant, perfume, hairspray, and insect repellant. The aerosol may be referred to as a spray or mist.

The compositions comprising ammonia oxidizing microorganisms, e.g., N. eutropha may also comprise one or more of a moisturizing agent, deodorizing agent, scent, colorant, insect repellant, cleansing agent, or UV-blocking agent.

In some embodiments, ammonia oxidizing microorganisms, e.g., N. eutropha are associated with cloth, yarn, or thread. Articles of clothing such as, for example, shoes, shoe inserts, pajamas, sneakers, belts, hats, shirts, underwear, athletic garments, helmets, towels, gloves, socks, bandages, and the like, may also be treated with ammonia oxidizing bacteria, e.g., N. eutropha. Bedding, including sheets, pillows, pillow cases, and blankets may also be treated with ammonia oxidizing bacteria, e.g., N. eutropha. In some embodiments, areas of skin that cannot be washed for a period of time may also be contacted with ammonia oxidizing bacteria, e.g., N. eutropha. For example, skin enclosed in orthopedic casts which immobilize injured limbs during the healing process, and areas in proximity to injuries that must be kept dry for proper healing such as stitched wounds may benefit from contact with the ammonia oxidizing bacteria, e.g., N. eutropha.

In some aspects, the present disclosure provides a wearable article comprising ammonia oxidizing microorganisms as described herein. A wearable article may be a light article that can be closely associated with a user's body, in a way that does not impede ambulation. Examples of wearable articles include a wristwatch, wristband, headband, hair elastic, hair nets, shower caps, hats, hairpieces, and jewelry. The wearable article comprising an ammonia oxidizing bacteria, e.g., N. eutropha strain described herein may provide, e.g., at a concentration that provides one or more of a treatment or prevention of a skin disorder, a treatment or prevention of a disease or condition associated with low nitrite levels, a treatment or prevention of body odor, a treatment to supply nitric oxide to a subject, or a treatment to inhibit microbial growth.

In some embodiments, the ammonia oxidizing microorganisms, e.g., N. eutropha are associated with a product intended to contact the hair, for example, a brush, comb, shampoo, conditioner, headband, hair elastic, hair nets, shower caps, hats, and hairpieces. Nitric oxide formed on the hair, away from the skin surface, may be captured in a hat, scarf or face mask and directed into inhaled air.

Articles contacting the surface of a human subject, such as a diaper, may be associated with ammonia oxidizing microorganisms, e.g., N. eutropha. Because diapers are designed to hold and contain urine and feces produced by incontinent individuals, the urea in urine and feces can be hydrolyzed by skin and fecal bacteria to form free ammonia which is irritating and may cause diaper rash. Incorporation of bacteria that metabolize urea into nitrite or nitrate, such as ammonia oxidizing bacteria, e.g., N. eutropha, may avoid the release of free ammonia and may release nitrite and ultimately NO which may aid in the maintenance of healthy skin for both children and incontinent adults. The release of nitric oxide in diapers may also have anti-microbial effects on disease causing organisms present in human feces. This effect may continue even after disposable diapers are disposed of as waste and may reduce the incidence of transmission of disease through contact with soiled disposable diapers.

In some embodiments, the product comprising ammonia oxidizing microorganisms, e.g., N. eutropha is packaged. The packaging may serve to compact the product or protect it from damage, dirt, or degradation. The packaging may comprise, e.g., plastic, paper, cardboard, or wood. In some embodiments the packaging is impermeable to bacteria. In some embodiments, the packaging is permeable to oxygen and/or carbon dioxide.

Methods of Treatment with Ammonia Oxidizing Microorganisms

In accordance with one or more embodiments, a subject may be treated via administration of ammonia oxidizing microorganisms, e.g., a preparation comprising ammonia oxidizing microorganisms. As used herein, treatment of a subject may comprise administering an ammonia oxidizing microorganism composition for a cosmetic or therapeutic result. For instance, treatment may comprise treating or alleviating a condition, symptom, or side effect associated with a condition or achieving a desired cosmetic effect.

Subjects may include an animal, a mammal, a human, a non-human animal, a livestock animal, or a companion animal. The subject may be female or male. The subject may have various skin types. The subject may have various health-related profiles, including health history and/or genetic predispositions. The subject may generally have a normal microbiome, e.g., a physiological microbiome, or a disrupted microbiome. The subject may be characterized as one of the following ethnicity/race: Asian, black or African American, Hispanic or Latino, white, or multi-racial. The subject may be of an age of less than 1, or between 1-5, 5-10, 10-20, 20-30, 30-40, 40-50, 50-60, or over 60 years.

The ammonia oxidizing microorganisms that may be used to treat a subject include all the ammonia oxidizing microorganisms, e.g., N. eutropha compositions described in this application, e.g. a purified preparation of optimized ammonia oxidizing microorganisms, for instance strain D23.

The methods may be provided to administer, or deliver a therapeutic product or a cosmetic product. The methods may comprise administering or introducing a preparation comprising live ammonia oxidizing microorganisms to a subject. The preparation may be formulated to treat a target indication and/or formulated for a desired mode of delivery.

In accordance with one or more embodiments, a preparation comprising live ammonia oxidizing microorganisms may be administered to a first tissue of a subject. The first tissue may be a deposit tissue. The first tissue may be a target tissue or a tissue other than a target tissue. The live ammonia oxidizing microorganisms, or a product thereof, e.g., nitrite and/or nitric oxide, may then move or be transported to a second tissue, e.g., via diffusion. The second tissue may be a target tissue. The target tissue may be associated with a desired local or systemic effect. The target tissue may be associated with an indication, disorder, or condition to be treated.

Ammonia oxidizing microorganism preparations may be administered, for example to the skin, for a cosmetic or therapeutic effect. For instance, administration may provide a cosmetic treatment, benefit, or effect. In some embodiments, administration may provide for treatment or improvement of one or more of oily appearance, pore appearance, radiance, blotchiness, skin tone evenness, visual smoothness, and tactile smoothness. In some embodiments, a cosmetic appearance of a subject may be altered such as may result from improved skin health. Signs of aging may be reduced, delayed, or reversed. Administration may result in a qualitative improvement in skin and/or scalp condition and/or quality. Skin smoothness, hydration, tightness, and/or softness in a subject may be improved. The present disclosure also provides a method of reducing body odor.

Administration may provide a therapeutic treatment, benefit, or effect. The present disclosure provides a method of supplying nitrite and nitric oxide to a subject. The present disclosure provides various methods for the suppression, treatment, or prevention of diseases, disorders, infections, and conditions using ammonia oxidizing microorganisms. Ammonia oxidizing microorganisms may be used, for instance, to treat various diseases associated with low nitrite levels, skin diseases, and diseases caused by pathogenic bacteria. In some embodiments, administration may provide for a reduction in inflammation. Indeed, a local or systemic anti-inflammatory effect may be demonstrated. In some non-limiting embodiments, inflammation may be downregulated. In at least some embodiments, microbial growth may be inhibited. Skin and overall health may be improved. Inadequate circulation may be augmented. Endothelial function may be promoted. A change in level of nitrite or NO at a target tissue or in circulation may be demonstrated. In some embodiments, administration, e.g., administration of an effective amount, may modulate, change, or alter a level of nitrite or NO at a target tissue or in circulation. In some embodiments, administration, e.g., administration of an effective amount, may result in an increased level of nitrite or NO at a target tissue or in circulation.

Administration of the compositions disclosed herein may provide transmucosal delivery and/or circulation, e.g. locally or systemically. In some embodiments, administration may provide that ammonia oxidizing microorganisms, products thereof, or byproducts thereof (e.g., nitrate, nitrite, NO, or CoQ8) penetrate a deposit or target tissue at least 10%, 20%, 30%, 40%, 50%, 60%, 70%, 80%, 90%, or 100%. In at least some embodiments, 10%, 20%, 30%, 40%, 50%, 60%, 70%, 80%, 90%, or 100% of ammonia oxidizing microorganisms, products thereof, or byproducts thereof, penetrate a deposit or target tissue or enter circulation upon administration of the compositions disclosed herein.

The preparations and methods of the present disclosure may provide for reducing an amount of undesirable microorganisms from an environment associated with a subject. The ammonia oxidizing microorganisms described herein may out-compete other organisms by, e.g., consuming scarce nutrients, or generating byproducts that are harmful to other organisms, e.g., changing a pH level that is not conducive to the undesirable organism's growth.

The present disclosure also provides a method of promoting wound healing, including of chronic wounds, such as in a patient that has an impaired healing ability, e.g., a diabetic patient. A bandage including ammonia oxidizing microorganisms may optionally be applied to the wound.

It is appreciated that many modern degenerative diseases may be caused by a lack of NO species, and that AOM may be administered to supply those species, directly to a target tissue or via diffusion to a target tissue. Application of AOM may resolve long standing medical conditions. In certain embodiments, AOM are applied to a subject to offset modern bathing practices, especially with anionic detergents which remove AOM from the external skin.

In accordance with one or more embodiments, AOM convert ammonia to nitrite, an anti-microbial compound, and nitric oxide, a well-documented signaling molecule in the inflammatory process.

The present disclosure provides, inter alia, a method of modulating a composition of a microbiome, e.g., modulating or changing the proportions of a microbiome in an environment, e.g., a surface, e.g., a surface of a subject. This may, in turn, exhibit a health-related benefit. The method may comprise administering a preparation comprising ammonia oxidizing microorganisms to a subject. In some embodiments, the amount and frequency of administration, e.g., application, may be sufficient to reduce a proportion of pathogenic microorganisms.

Application of ammonia oxidizing microorganisms to a subject, e.g., a human subject may lead to unexpected changes in the microbiome. It may lead to increases in the proportion of normal commensal non-pathogenic species and reductions in the proportion of potentially pathogenic, pathogenic, or disease causing organisms.

An increase in the proportion of non-pathogenic bacteria may occur with a pre-determined period of time, e.g., in less than 1 day, 2 days, 3 days, 4 days, 5 days, 1 week, 2 weeks, 3 weeks, or 4 weeks, or in less than 1-3, 3-5, 5-7, 7-9, 5-10, 10-14, 12-18, 12-21, 21-28, 28-35, 35-42, 42-49, 49-56, 46-63, 63-70, 70-77, 77-84, 84-91 days.

A decrease in the proportion of pathogenic bacteria may occur with a pre-determined period of time, e.g., in less than 1 day, 2 days, 3 days, 4 days, 5 days, 1 week, 2 weeks, 3 weeks, or 4 weeks, or in less than 1-3, 3-5, 5-7, 7-9, 5-10, 10-14, 12-18, 12-21, 21-28, 28-35, 35-42, 42-49, 49-56, 46-63, 63-70, 70-77, 77-84, 84-91 days.

In accordance with one or more embodiments, a subject may be evaluated for need of treatment. In some embodiments, a subject may be selected on the basis of the subject being in need of a treatment. The present disclosure may further provide obtaining a sample from a subject and analyzing the sample. In some embodiments, subjects may be evaluated before, during, and/or after treatment, such as at predetermined time intervals.

In accordance with one or more embodiments, administration may be performed before, during, or subsequent to occurrence of a health-related condition, or in response to a warning sign, trigger, or symptom thereof. In accordance with one or more embodiments, a second amount of the preparation may be administered to the subject, e.g., a second dose.

In certain aspects, the present disclosure provides combination therapies comprising ammonia oxidizing microorganisms, e.g., a N. eutropha and a second treatment, e.g. a therapeutic. For instance, the disclosure provides physical admixtures of the two (or more) therapies are physically admixed. In other embodiments, the two (or more) therapies are administered in combination as separate formulation. The second therapy may be, e.g., a pharmaceutical agent, surgery, diagnostic, or any other medical approach that treats, e.g., is approved to treat or commonly used to treat, the relevant disease, disorder, or a symptom of the relevant disease or disorder. The second treatment may be administered before or after the administration. The effective amount can be administered concurrently with the second treatment. The second treatment may be administered via the same or a different mode of delivery. The subject may have a therapeutic level of the second treatment upon administration of the preparation. In certain embodiments, the second treatment may provide an anti-inflammatory effect or be administered to reduce inflammation at the target site. In at least some embodiments, the preparation may be administered concurrently or in conjunction with a product or byproduct of the ammonia oxidizing microorganisms, e.g., nitrite, nitrate, nitric oxide, CoQ8. In at least some embodiments, the preparation may be administered concurrently or in conjunction with a composition that promotes growth or metabolism of ammonia oxidizing microorganisms, promotes production of products or byproducts of ammonia oxidizing microorganisms, promotes urease activity, or has a synergistic effect with ammonia oxidizing microorganisms, e.g., ammonia, ammonium salts, urea, and urease.

The preparation may be administered with a microbiome cleansing preparation, for example a local or systemic antibiotic. The preparation may be administered after administration of a cleansing preparation or a bowel cleanse. The preparations may be administered pre- or post-surgical procedure, diagnostic procedure, or natural event, e.g., giving birth. The preparations may be administered before, during, or after deposit of an implantable or invasive device.

In accordance with one or more embodiments, the preparation may be administered as an analgesic or prophylactic. The preparation may be self-administered. The administration of the preparation may be device-assisted.

In some embodiments, the ammonia oxidizing microorganisms, e.g., a preparation of ammonia oxidizing microorganisms, are administered at a dose of about or greater than about 10³-10⁴ CFU, 10⁴-10⁵ CFU, 10⁵-10⁶ CFU, 10⁶-10⁷ CFU, 10⁷-10⁸ CFU, 10⁸-10⁹ CFU, 10⁹-10¹⁰ CFU, 10¹⁰-10¹¹ CFU, 10¹¹-10¹² CFU, 10¹²-10¹³ CFU, or 10¹³-10¹⁴ CFU per application, per day, per week, or per month. In some embodiments, the ammonia oxidizing microorganisms are administered at a dose of about 10⁹-10¹⁰ CFU, e.g., about 1×10⁹-5×10⁹, 1×10⁹-3×10⁹, or 1×10⁹-10×10⁹ CFU per application or per day.

In some embodiments, the ammonia oxidizing microorganisms are administered in a volume of about 1-2, 2-5, 5-10, 10-15, 12-18, 15-20, 20-25, or 25-50 ml per dose. In some embodiments, the solution is at a concentration of about 10⁸-10⁹, 10⁹-10¹⁰, or 10¹⁰-10¹¹ CFU/ml. In some embodiments, the ammonia oxidizing microorganisms are administered as two 15 ml doses per day, where each dose is at a concentration of 10⁹ CFU/ml.

In some embodiments, the ammonia oxidizing microorganisms are administered once, twice, three, or four times per day. In some embodiments, the ammonia oxidizing microorganisms is administered once, twice, three, four, five, or six times per week. In some embodiments, the ammonia oxidizing microorganisms is administered shortly after bathing. In some embodiments, the ammonia oxidizing microorganisms is administered shortly before sleep.

In some embodiments, the ammonia oxidizing microorganisms are administered for about 1-3, 3-5, 5-7, 7-9, 5-10, 10-14, 12-18, 12-21, 21-28, 28-35, 35-42, 42-49, 49-56, 46-63, 63-70, 70-77, 77-84, 84-91 days, e.g., for about 1 month, for about 2 months, for about 3 months. In some embodiments, the ammonia oxidizing microorganisms is administered for an indefinite period of time, e.g., greater than one year, greater than 5 years, greater than 10 years, greater than 15 years, greater than 30 years, greater than 50 years, greater than 75 years.

### Administration of Ammonia Oxidizing Microorganisms to the Gastrointestinal System

The formulations (e.g., preparations or compositions) described herein may include those suitable for enteral delivery, e.g., oral administration, sublingual, and rectal administration. Ammonia oxidizing microorganism preparations may be administered to the gastrointestinal system for cosmetic or therapeutic purposes. For instance, compositions include those formulated for cosmetic or therapeutic use.

The enteral formulations (e.g., preparations or compositions) may conveniently be presented in unit dosage form and may be prepared by any of the methods known in the art of pharmacy or cosmetology. Typically, methods include the step of bringing the active ingredient (e.g., ammonia oxidizing microorganism) into association with a pharmaceutical carrier which constitutes one or more accessory ingredients. In general, the pharmaceutical or cosmetic formulations are prepared by uniformly and intimately bringing into association the active ingredient with liquid carriers or finely divided solid carriers or both and then, if necessary, shaping the product into the desired formulation.

Enteral formulations may be presented as discrete units, each containing a predetermined amount of the active ingredient as a solution or suspension in an aqueous or non-aqueous liquid, as a powder or granules, or as an oil-in-water or water-in-oil liquid emulsion. Various pharmaceutically acceptable carriers and their formulations are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2 S, 1988; Aulton, M. and Taylor, K., Aulton's Pharmaceutics: The Design and Manufacture of Medicines, 5^{th} Edition, 2017; Antoine, A., Gupta M. R., and Stagner, W. C., Integrated Pharmaceutics: Applied Preformulation, Product Design, and Regulatory Science, 2013; Dodou K. Exploring the Unconventional Routes—Rectal and Vaginal Dosage Formulations, The Pharmaceutical Journal, 29 Aug. 2012.

Compositions disclosed herein may be prepared in enteral dosage formulations. Ammonia oxidizing microorganisms can administered enterally for cosmetic or therapeutic purposes. Enteral formulations are generally intended for absorption through the various epithelia and mucosa of the gastrointestinal system. For instance, compositions may be prepared as tablets, capsules, solutions, suspensions, emulsions, or suppositories. Each of the dosage forms may be formulated to comprise one or more carrier or excipient, as described in more detail below. Solid dispersion forms may comprise tablets and capsules. Tablets may be formulated to break up into granules and powders once in the gastrointestinal tract. Capsules may be formulated as soft shell or hard shell capsules. Liquid dispersion forms, e.g., oral dispersion forms, may comprise solutions, suspensions, or emulsions of the active agent in a vehicle.

Compositions prepared for rectal administration may be formulated the same or differently as oral tablets, capsules, solutions, suspensions, and emulsions. However, rectal compositions may further be formulated for administration as rectal solutions, suppositories, ointments, gels, emulsions, or films. Typically, rectal solutions may be aqueous solutions, e.g., an aqueous dispersion of the active agent. Rectal ointments may comprise anhydrous dispersions of the active agent, e.g., in a mineral oil-white petroleum base. Rectal gels may comprise a polymer, e.g., poloxamers, xanthan gum, gellan gum, locust bean gum, and carrageenan. Rectal ointments and gels may provide for a longer residence time than, for example, aqueous solutions. The longer residence time may further allow for a reduced dosing interval. Rectal emulsions may comprise microspheres, microcapsules, nanoparticles, nanocapsules, micelles, liposomes, niosomes, dendrimers, or cyclodextrin complexes. Rectal films, e.g., may comprise a water-soluble polymeric film or a polyvinyl alcohol polymeric film that dissolves when in contact with bodily fluids, releasing the active agent.

Ammonia oxidizing compositions disclosed herein may comprise an effective amount of AOMs, for example, to increase mucus thickness in at least a portion of the gastrointestinal system, to colonize a tissue of the gastrointestinal system, to treat a gastrointestinal disorder or a symptom of a gastrointestinal disorder, to improve digestion in a subject, or to promote endothelial function, e.g., within the gastrointestinal system. The compositions may be administered on a substantially empty stomach or after a bowel cleanse or antibiotic treatment. In some embodiments, water is administered to the subject after administration of the ammonia oxidizing microorganism composition. In some embodiments, several hours are waited prior ot food consumption after administration. Such compositions may be formulated for topical, oral, sublingual, sublabial, buccal, rectal, or device-assisted application. Topical application formulations may include, e.g., enema, douche, wash, spray, aerosol, and mist. Oral application formulations are generally prepared specifically for ingestion through the mouth. Sublingual and sublabial formulations, e.g., tablets, strips, drops, sprays, aerosols, mists, lozenges, and effervescent tablets, may be administered orally for diffusion through the connective tissues under the tongue or lip. Specifically, formulations for sublingual administration may be placed under the tongue and formulations for sublabial administration may be placed between the lip and gingiva (gum). Sublabial administration may be beneficial when the dosage form comprises materials that may be corrosive to the sensitive tissues under the tongue. Buccal formulations may generally be topically held or applied in the buccal area to diffuse through oral mucosa tissues that line the cheek. Sublingual, sublabial, and buccal administration may provide a more rapid onset of action as compared to oral administration because the active agent directly enters the bloodstream, avoiding first pass metabolism. Rectal application may be achieved by inserting the formulation in the rectal cavity, either with or without the assistance of a device. Device-assisted application may include, for example, delivery via an applicator or an insertable applicator, catheter, feeding tube, or delivery in conjunction with an endoscope or ultrasound. Suitable applicators include liquid formulation bulbs and launchers and solid formulation insertable applicators.

The time of onset of action for the formulations disclosed herein may be dependent on the formulation and may range from seconds to minutes to hours. For example, tablets and rectal solid dosage forms may provide action within minutes. Buccal tablets may provide action within minutes. Suppositories, solutions, and suspensions may provide action within minutes or hours. Powders, granules, tablets, and capsules may provide action within minutes to hours. Modified release tablets may provide action within minutes to hours. Gastro-resistant coated formulations may provide action within hours. The release time for the formulations disclosed herein may be dependent on the formulation and may range from minutes to hours to days. For example, the dosage forms may be formulated to provide fast-release within minutes or extended release within hours. Certain dosage forms may provide extended release within days or months.

Ammonia oxidizing microorganism compositions disclosed herein may be in the form of a tablet or capsule. Tablets may generally comprise disintegrants to promote tablet break-up into smaller particles, e.g., granules and powder particles, facilitating dissolution and absorption. Tablets may be coated to provide a protective barrier to environmental factors for drug stability, mask unpleasant drug taste, or protect drugs from acidic conditions in the stomach. Tablets may further comprise fillers, binders, bulking agents, and diluents to increase bulk of the tablet, binders to promote cohesion, antiadherents and antisticking agents to decrease filming, adherence, and sticking, glidants and flow aids to decrease interfacial cohesion and friction, wetting agents, solubilizing agents, stabilizers, colorants, sweeteners, and flavors. Capsules may generally comprise a filler, e.g., a liquid, gel, semi-liquid, semi-solid, or micro-emulsion formulation, and a coating, e.g., a hard shell or soft shell coating, comprising gelating, hypermellose, or polymeric material. Hard shell capsules may be filled with solids (e.g., powders, granules, pellets, tablets, and capsules), semi-solids (e.g., gels, pastes, and thermosetting polymers), and liquids (e.g., solutions, suspensions, emulsions, and micro-emulsions). Soft shell capsules may be filled with liquids and semi-liquids. Capsules may comprise plasticizers (e.g., glycerin, sorbitol, propylene glycol, poly(ethylene glycol), hydrogenated saccharides, polysaccharides, and sorbitan) to decrease brittleness, processing aids to improve gelling and settling (e.g., carrageenan and cations), buffers, surfactants, wetting agents, lubricants, colorants, opacifying agents, and flavors. The coating may readily rupture or dissolve following administration. Tablets and capsules may further comprise modified-release products, e.g., fast-dissolving for immediate delivery or controlled, delayed, or sustained release, e.g., polymeric based components or coating membranes.

Compositions disclosed herein may be specifically formulated to be gastro-resistant, for example, to be resistant to the harsh and acidic environment of the stomach. In some embodiments, ammonia oxidizing microorganism compositions are comprised in a gel or agar matrix that is formulated to withstand acidic environments (e.g., the acidic environment of the stomach) and break down in the physiological pH of the intestines. Formulations may be coated with a composition that is formulated to withstand certain gastrointestinal environments and deliver the active agent, for example, by breaking down, in the physiological environment associated with a specific gastrointestinal tissue.

Ammonia oxidizing microorganism compositions may be in the form of a liquid or semi-liquid dispersion forms, for example a syrup or linctus. Liquid and semi-liquid dispersions may comprise a vehicle for delivery, density modifiers to increase density of a dispersion medium, for example, to match the density of dispersed particles, and viscosity modifiers to increase viscosity and decrease sedimentation. Dispersions may comprise wetting agents and surfactants to decrease interfacial energy between the vehicle and dispersed particles, flocculating agents to control the level of loose aggregate or floc formulation, humectants to decrease the rate of product moisture loss, and buffers, sweeteners, flavoring, and colorants as disclosed herein.

Exemplary compositions include suspensions which can contain, for example, microcrystalline cellulose for imparting bulk, alginic acid or sodium alginate as a suspending agent, methylcellulose as a viscosity enhancer, dicalcium phosphate, starch, magnesium stearate and/or lactose and/or other excipients, binders, extenders, disintegrants, diluents and lubricants, mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, glidants, flavors, coloring agents and stabilizers may also be added for ease of fabrication and use. The surfactant may be a zwitterionic surfactant, a non-ionic surfactant, or an anionic surfactant.

Suppositories include solid dosage forms intended for introduction into the rectal cavity. The suppository may melt, releasing the active agent, once introduced into the rectal cavity. The rate of delivery of the active agent may be influenced by selection of pharmaceutically acceptable carrier or suppository base. Suitable suppository bases include fatty base and water base. Suitable fatty base formulations may comprise theobroma oil (cocoa butter), spermaceti (beeswax), synthetic triglycerides or triglycerides from hydrogenated vegetable oils, palm, palm kernel, or coconut oils. Suitable water base formulations may comprise glycerinated gelatin or polyethylene glycol polymers. Suppository formulations may further comprise an absorption enhancer.

Solid rectal dosage forms comprising the compositions disclosed herein may be formulated as molded tablets, compressed tablets, or capsules. Molded tablets may be made in a variety of shapes, e.g., cone shaped, and prepared in a similar way to suppositories. Compressed tablets may be made in a variety of shapes and prepared by compression. Compressed rectal tablets may typically comprise similar formulations and excipients as oral tablets. Accordingly, compressed rectal tablets disclosed herein may be formulated to include one or more of fillers, binders, bulking agents, diluents, disintegrants, lubricants, anti-adherents and anti-sticking agents, glidants and flow agents, wetting agents, solubilizing agents, drug-release modifiers, stabilizers, and colorants. Rectal capsules may be prepared in a manner similar to gelatin oral capsules. For instance, rectal capsules disclosed herein may be formulated to include one or more of solid, semisolid, and liquid fillers, plasticizers, processing aids, surfactants, colorants, opacifying agents, and preservatives. Rectal capsules body may comprise gelatin, hypermellose, hydroxypropyl methylcellulose, hydroxypropyl starch, starch modifications, and pullulan.

Ointments, foams, and gels may generally be formulated to be more viscous than aqueous solutions and provide for a longer residence time within a body cavity, e.g., the rectal cavity. Such viscous liquid formulations may comprise a gel or gelling agent. For instance, a gelling agent may be a thermoreversible gel. A thermoreversible gel may be a liquid at lower or room temperature and turn to gel once inserted into the body cavity, e.g., rectum or colon. The gel or gelling agent may allow easier administration and positioning of the dosage form. For instance, the gel or gelling agent may prevent the dosage form from leaking out of the body cavity. Thermoreversible polymers include poloxamer. Mucoadhesive polymers include sodium alginate. Gels or gelling agents may further comprise a solubility enhancer, for example, hydroxypropyl-betalcyclodextrin. Gel formulations may include, but are not limited to agar, silica, polyacrylic acid (for example Carbopol®), carboxymethyl cellulose, starch, guar gum, alginate or chitosan.

Solutions containing the compositions disclosed herein may be formulated as, e.g., enemas or douches for rectal delivery. Generally, enemas or douches may be aqueous solutions comprising the active agent. Enemas may be administered to reach a deep rectal cavity, e.g., the colon, or a superficial rectal cavity, e.g., the rectum. In some embodiments, enemas are administered in volumes of 2 L or less. The U.S. Department of Health and Human Services has discouraged the use of douches, citing several risks, including irritation, bacterial infection, and urogenital inflammatory diseases. However, in some certain situations, physicians may still order douches for medical reasons. Because antiseptics used during douches may disturb the natural balance of microorganisms in the body cavity, causing infections, such physician-ordered douches may be administered in combination with the ammonia oxidizing microorganism compositions disclosed herein. Furthermore, the ammonia oxidizing microorganism compositions disclosed herein may be administered in a douche as a primary or secondary therapy, for example, in combination with one or more additional treatments. Additionally, solutions may be formulated as a spray, aerosol, or mist for topical delivery to the rectal area.

Exemplary compositions may include one or more excipients, for example, absorption and penetration enhancers, analgesics, local analgesics, antifungal agents, anti-inflammatory agents, steroids and corticosteroids, thermoreversible gels, preservatives, antioxidants, buffers, chelating agents, ion exchange agents, solubilizing agents, suspending agents, thickeners, surfactants, wetting agents, tonicity-adjusting agents, and a vehicle for proper drug delivery. Absorption and penetration enhancers may improve the ability of the active agent to be absorbed by a number of different mechanisms. Analgesics and local analgesics may be used to relieve pain and/or decrease subject discomfort. Steroids and corticosteroids may help reduce inflammation. Thermoreversible gels may improve positioning and retention time of the active agent. Antioxidants may reduce the oxidative degradation of the active agent. Buffers may maintain a desired pH of the composition and/or enhance solubility or stability of the composition. Chelating agents may include complex trace metals that catalyze oxidation reactions of the composition. Ion exchange agents may control the release of active agent by ion exchange mechanisms. Solubilizing agents may increase the solubility of the active agent or another excipient. Suspending agents and thickeners may increase the viscosity or density of the composition to increase the active agent's retention time and residence time in the gastrointestinal system, for example the oral or rectal cavity. Surfactants, including cationic, anionic, and non-ionic surfactants, and wetting agents may act to wet insoluble hydrophobic active agent or other excipients. Tonicity-adjusting agents may provide an isotonic solution with urogenital fluids. Vehicle, for example water or fatty base, may provide bulk for proper active agent delivery.

Excipients that can be included are, for instance, proteins, such as human serum albumin or plasma preparations. If desired, the pharmaceutical composition may also contain minor amounts of non-toxic auxiliary substances, such as wetting or emulsifying agents, preservatives, and pH buffering agents and the like, for example sodium acetate or sorbitan monolaurate. In some embodiments, excipients, e.g., a pharmaceutically acceptable excipient or a cosmetically acceptable excipient, may comprise an anti-adherent, binder, coat, disintegrant, filler, flavor, color, lubricant, glidant, sorbent, preservative, or sweetener. In some embodiments, the preparation may be substantially free of excipients. In some embodiments, the preparation may be substantially free of one or more of the compounds or substances listed in the disclosure.

The ammonia oxidizing microorganism compositions can, for example, be administered in a form suitable for immediate release or extended release. Suitable examples of immediate release formulations include topical formulations, oral formulations, buccal formulations, sublingual formulations, sublabial formulations, and rectal delivery formulations. Topical formulations for immediate release may include, e.g., solutions, suspensions, emulsions, foams, gels, and ointments. Topical formulations may be formulated for immediate release to avoid complications from clearance by bodily fluids, e.g., in the rectal cavity. Rectal delivery formulations for immediate release include, e.g., suppositories, rectal solid forms, and films. Each of the rectal delivery formulations may be formulated to experience a phase change upon coming into contact with bodily fluids within the body cavity and release the active agent. For instance, suppositories and tablets may be formulated for immediate release to avoid challenges caused by dosage form expulsion and low adhesion to the rectal cavity membrane. Certain target membranes, for example, the rectal mucosa, allow for quick active agent absorption, while rich localized vasculature enables easy update to systemic circulation. Oral delivery formulations for immediate release include liquid dispersion forms, tablets, and capsules. Specifically, buccal, sublabial, and sublingual formulations may provide immediate release of the active agent, for example into systemic circulation, by facilitating uptake through the oral mucosa, sublingual, and sublabial tissues, which may bypass first pass metabolism.

Controlled release oral formulations may be prepared as liquid dispersion or solid dispersion forms. Suitable examples of sustained-release systems include suitable gelating or polymeric materials (polymer based cores or coating membranes), for example semi-permeable polymer matrices in the form of shaped articles, e.g., films, or microcapsules; suitable hydrophobic materials, for example as an emulsion in an acceptable oil; or ion exchange resins. The pharmaceutical compositions may be in the form of particles comprising one or more of biodegradable polymers, polysaccharide jellifying and/or bioadhesive polymers, or amphiphilic polymers. These compositions exhibit certain biocompatibility features which allow a controlled release of an active substance.

In some embodiments, controlled release rectal formulations may be formulated as an ointment, gel, foam, or emulsion. Pharmaceutical extended release compositions, e.g., rectal delivery compositions, may be formulated with one or more mucoadhesive agent, e.g., mucoadhesive gel or dry mucoadhesive tablet. The mucoadhesive agent may aid in attachment to the rectal body cavity, e.g., rectal mucosa. Similarly, solid dosage forms e.g., suppositories, tablets, capsules, or films, may be formulated with one or more mucoadhesive agent which may enhance dosage form positioning within the rectal cavity or may remain present when part of the solid dosage form melts or disintegrates. For instance, a solid dosage form may be formulated to dissolve rapidly when in contact with bodily fluid and turn to a mucoadhesive viscous solution that attaches to the oral mucosa or rectal cavity mucosa and is gradually washed out without requiring removal. Sublabial dosage forms may provide controlled or extended release of the active agent. For instance, sublabial administration between the upper lip and gum may prevent the preparation from being swallowed with salivation, permitting slow release of the drug directly to the connective tissues and associated vasculature over a long period of time.

In some non-limiting embodiments, the preparations may be one or more of: substantially odorless, colorless, not associated with substantial side effects, non-toxic, well-tolerated, have no adverse effects if released into the environment, no risk of fostering antibiotic resistance, and have a physiology such that it can interact positively with various human gut microbiomes under normal and disease states.

The ammonia oxidizing microorganism compositions can, for example, be administered in form suitable to provide local treatment or systemic treatment. Local effects may be achieved, for example, by rapid absorption through the tissues of the gastrointestinal system, and systemic effects may be achieved, for example, by drug absorption through various epithelia and mucosa of the gastrointestinal tissues. Compositions disclosed herein may be administered to treat a local inflammatory disease, a symptom of a local or systemic inflammatory disease, or a side effect caused by a local or systemic inflammatory disease. Suitable examples of local gastrointestinal system conditions or gastrointestinal disorders that may be treated with compositions disclosed herein include, irritable/inflammatory bowel syndrome, Crohn's disease, colitis, nectrotizing enterocolitis, ulcers, ulcerative colitis, Celiac's disease, gluten sensitivity, lactose intolerance, food and/or beverage intolerance, heartburn, acid reflux, dyspraxia, small intestinal bacterial overgrowth (SIBO), pancreatitis, appendicitis, gastritis, malabsorption disorders, and gasteroenteritis. Leaky gut syndrome can be addressed. Administration may be used to promote GI health. A gastrointestinal state of a subject may be impacted, e.g., a composition of a GI microbiome may be changed, altered, or modulated, such as by changing proportions of microorganisms therein, such as in the gut or GI tract. Administration may be used prior to travel to reduce the likelihood of GI upset upon exposure to unfamiliar food, drink, and/or microorganisms. In some embodiments, ammonia oxidizing microorganism compositions can be administered in a form suitable to treat certain infections and inflammatory disorders, e.g., bacterial infections, fungal infections, viral infections, itching, local inflammation, and wound healing. For instance, ammonia oxidizing microorganism compositions may be administered to treat inflammation associated with a surgical or diagnostic procedure, dental treatment, catheter-based transfers (e.g., matter transfers in, out, or in between two locations within the body), enteral nutrition (e.g., feeding tube), stents, or generally inflammation related to any foreign body introduced into the gastrointestinal or biliary system. Ammonia oxidizing microorganisms may be administered to treat localized symptoms of gastrointestinal conditions, disorders, or systemic disorders, e.g., stomach pain, stomach cramping, gas, constipation, discomfort, change in bowel habit, and diarrhea, or side effects associated with such conditions, disorders, or systemic disorders, e.g., hyperammonemia, and biliary system disorders, e.g., liver failure and acute liver failure. In at least some embodiments, administration of ammonia oxidizing microorganism compositions may reduce a symptom or side effect associated with a gastrointestinal condition or disorder, e.g., bloating, diarrhea, gas, stomach pain, stomach cramping, or borborygmus.

Examples of systemic conditions that may be treated with compositions disclosed herein include headaches, cardiovascular diseases, connective tissue disorders, inflammation, immune responses and autoimmune disorders, liver diseases, infections, neurological diseases, psychiatric disorders, nitric oxide disorders, urea cycle disorders, congestion, vasodilation disorders, skin diseases, wound healing, bowel disorders, reactions to insect bites, ophthalmic disorders, and certain viral, bacterial, and fungal infections. For instance, systemic conditions that may be treated with compositions disclosed herein include cardiovascular diseases such as cardioprotection, heart failure, hypertension, pulmonary arterial hypertension; immune responses and autoimmune disorders such as alopecia and vitiligo; liver diseases such as non-alcoholic fatty liver disease (NAFLD), non-alcoholic steatohepatitis (NASH); neurological diseases and psychological disorders such as depression, insomnia, and diabetic neuropathy; nitric oxide disorders such as erectile dysfunction; wound healing, e.g., from bed sores and nursing home care, burns, diabetic ulcers e.g., foot ulcer, venous leg ulcer, biofilm, and mouth sores; skin diseases and disorders such as hyperhydrosis, pruritus, helomas, and subtypes of helomas; ophthalmic disorders such as blepharitis, dry eye, macular degeneration, and glaucoma; bowel disorders such as gluten sensitivity, irritable/inflammatory bowel disease, Crohn's disease, colitis, and necrotizing enterocolitis; and vasodilation disorders such as Renaud's disease, thermoregulation, and migraines. Certain viral, bacterial, and fungal infections may be treated with formulations disclosed herein, including infections caused by human papillomavirus (HPV), yeast infections, tinea versicolor, tinea unguium, tinea pedis/fungus, tinea cruris, jock itch, onychomycosis, dandruff, athlete's foot, sinusitis, otitis media, Methicillin-resistant Staphylococcus aureus (MRSA), staph, and bacterial vaginosis. Additional systemic conditions that may be treated with compositions disclosed herein include systemic inflammation, such as eczema, e.g., adult and pediatric eczema, hives, idiopathic uriticaria, lichen planus, insect bites including allergic reactions to insect bites, e.g., mosquito and Demodex folliculorum mite, reactions to poison ivy, itchiness, keratosis pilaris, laryngitis, pemphigus, psoriasis, rosacea, folliculitis and subtypes of folliculitis, hidradenitis supportiva, perioral dermatitis, lupus rash, seborrheic dermatitis, e.g., adult and infantile seborrheic dermatitis, acne, e.g., adolescent acne, adult acne, and cystic acne, diaper rash, occupational hand dermatitis, sunburn, and dermatomyositis. Additionally, compositions disclosed herein may be delivered or applied to treat certain cosmetic indications, including but not limited to, contact dermatitis, diaper odor, e.g., adult and pediatric, body odor, feminine odor, flaking, nail hardness, body odor, oily skin, razor burn, skin appearance, skit blotchiness, skin hydration, and sun spots. Compositions disclosed herein may be applied as a bug repellant or an antimicrobial agent.

Compositions disclosed herein may further be formulated as combination therapies. For instance, tablets or capsules may comprise distinct sections formulated for combination therapy. Initial and subsequent therapeutic treatments may be provided in a single dosage form, prepared in individual dosage forms, administered concurrently, or administered separately. Individual dosage forms may be administered via the same mode of administration, e.g., through the gastrointestinal system, or via an alternate mode of administration, e.g., orally, intranasally, topically, ocularly, via the auditory system, via the urogenital system, via the respiratory system, or via injection. For instance, combination therapies may comprise ammonia oxidizing microorganisms for treatment of an inflammatory disease or condition. Individual dosage forms may be administered by a surgical or diagnostic procedure. Individual dosage forms may be administered in combination with a surgical or diagnostic procedure. In some embodiments, ammonia oxidizing microorganism compositions, for example prepared for enteral administration, are formulated for combination therapy with an anti-inflammatory. Generally, compositions disclosed herein may be formulated for combination therapy with a drug or compound approved or commonly used to treat a disease, disorder, condition, symptom thereof, or side-effect thereof, for example a gastrointestinal disease, disorder, condition, symptom thereof, or side-effect thereof. In at least some embodiments, preparations formulated for administration in combination with a fecal matter transplant (FMT). Preparations may be formulated for administration in combination with a bowel cleanse or antibiotic to “plow the field” and allow for ammonia oxidizing microorganism colonization.

Preparations for administration to the gastrointestinal system may be formulated for targeted delivery to a specific deposit tissue or target tissue. In some embodiments, a preparation may be administered to a first tissue such that the preparation or a product of the preparation, e.g., ammonia oxidizing microorganisms or nitric oxide, is transported to a second tissue. The first tissue may be a deposit tissue. The second tissue may be a target tissue. The deposit tissue and the target tissue may be the same or different tissues. In some embodiments, the deposit tissue, the target tissue, or both may be a tissue of the gastrointestinal system. The preparation or product of the preparation may be delivered locally or systemically, e.g., at a deposit or target tissue or in circulation.

Preparations for gastrointestinal administration may be formulated for targeted delivery to a specific gastrointestinal tissue. Gastrointestinal deposit or target tissues include the mouth, stomach, salivary gland, oral cavity, pharynx, tongue, esophagus, liver, gallbladder, common bile duct, colon (transverse, ascending, and/or descending), cecum, appendix, rectum, anus, pancreas, pancreatic duct, large intestine, small intestine (duodenum, jejenum, and/or ileum), parotid, submandibular tissue, and sublingual tissue. In some embodiments, systemic update within the stomach may be problematic due to the acidic environment of the stomach. Systemic uptake within the liver may be problematic due to the metabolisation of compounds that enter the liver. Compositions may be specially formulated to bypass the stomach and/or liver and deliver the active agent to another target tissue. For instance, compositions may be formulated to deliver the target agent within the oral cavity or lower rectum, bypassing liver metabolism, or may be specially formulated to withstand the acidic environment of the subject and deliver the active agent to the small or large intestines. Generally, the gastrointestinal route may provide for systemic effects through the large available surface area and blood supply of gastrointestinal tissues.

The rectal route can be useful in pediatric and geriatric groups and patients who are unable to take oral medications due to nausea, vomiting, and unconsciousness. Rectal administration may be utilized to deliver drugs for preventing pre- and post-operative infections and treating inflammation. Preparations for rectal administration may be formulated for targeted delivery to a specific rectal tissue. Rectal deposit or target tissues may include superficial tissues, e.g., buttocks, anus, and the areas surrounding the anus, and internal tissues, e.g., rectum, colon, large intestine, small intestine, and anal sphincter muscles. Preparations for rectal administration may further be formulated for targeted delivery to a nearby tissue, e.g., pelvic floor muscles and prostate. Targeted delivery to a desired rectal tissue may be crucial in providing adequate treatment. Rectal dosage forms for targeted delivery to the upper rectum and colon, for example, may be absorbed by superficial rectal veins and delivered to the liver. Thus, rectal dosage forms for targeted delivery to the upper rectum and colon may be formulated for metabolism by the liver. Rectal dosage forms for targeted delivery to the lower rectum may be absorbed through inferior local veins and bypass the liver. Formulations that are not suitable for liver metabolism may be prepared for targeted delivery to the lower rectum.

In accordance with one or more embodiments, that AOM are able to grow and proliferate in the environment of the gastrointestinal system of a subject is surprising. The stomach, for example, may generally be characterized as anaerobic in nature. In some embodiments, there may be a transition to a higher pH level in the mucus at the stomach wall. In some embodiments, AOM may surprisingly act as a keystone species in regulating mucus thickness in the gastrointestinal system. Without wishing to be bound to any particular theory, nitrite levels may play a role in regulating mucus thickness in the GI system. Mucus may play an important barrier role in preventing, for example, infection within the GI system. In some non-limiting embodiments, the mucus layer may comprise two domains, a tightly adherent bacteria-free inner layer, such as may be indirect contact with a gut wall, and a somewhat less tight layer with a lower density of gel-forming peptides. In some non-limiting embodiments, mucus release may be increased which may, for example, facilitate digestion of disulfide containing substrates, including gluten. Protein disulfide isomerase may also be released. In some embodiments, thicker mucus may better seal the GI system wall, e.g., gut wall, and may reduce inflammation due to facultative anaerobes. In some non-limiting embodiments, Helicobacter pylori may be reduced by AOM colonization.

In at least some embodiments, the effects of superoxides may be counteracted. In some embodiments, AOM may suppress undesirable aerobes and facultative anaerobes therein. In some embodiments, mucus thickness may be increased while suppressing facultative anaerobes by depriving them of oxygen and nitrate. In at least some embodiments, AOM may live in mucus attached to the wall of the GI system. In accordance with one or more embodiments, AOM may surprisingly proliferate despite their long doubling time in the transient environment of the GI system. In some embodiments, AOM may surprisingly not promote diarrhea and rapid or uncontrollable emptying of the GI system, despite their production of NO and nitrite which may relax smooth muscle of the GI system and have this effect. In some embodiments, AOM may reduce constipation, for example, by improving the matching of peristalsis with nutrient absorption. In some embodiments, combination therapies may involve heterotrophic probiotics, such as Lactobacilli. In accordance with one or more embodiments, AOM may produce physiologically beneficial and/or significant levels of NO, nitrite, and/or NO precursors from ammonia and oxygen in the GI system. Without wishing to be bound by any particular theory, the GI system of a subject may be characterized by various oxygen and/or NO gradients. In at least some embodiments, colonizing the GI system with AOM may slow or stop the progression of various GI conditions, local and systemic, before they even manifest to optimize good health. In some embodiments, restoration of AOM following alcohol consumption may reduce and/or prevent some adverse effects, such as in the splanchnic system and liver.

In accordance with one or more embodiments, blood flow from the stomach passes through the liver. Without wishing to be bound by particular theory, nitric oxide may be the normal regulator of blood flow, mitochondria biogenesis, ATP status, and allocation of resources to immediate consumption or to repair. Inflammatory conditions in the GI system may be associated with adverse liver effects such as portal hypertension, primary sclerosing cholangitis, non-alcoholic steatohepatitis, non-infectious hepatitis and also multiple systemic inflammatory conditions such as asthma, Multiple sclerosis, chronic renal disease, psoriasis, pericarditis, and arthritis. Similarly, diabetes, stroke, and heart disease may be associated with chronic inflammatory disorders of the gut. Expanded blood volume and/or hyperdynamic circulatory state, such as in the splanchnic system may result. Decreased renal blood flow may be caused which may lead to kidney failure. Prevention of GI system inflammation by colonization with AOM in accordance with various embodiments may also reduce the incidences of liver disorders associated with GI inflammation and also other systemic inflammatory conditions. In at least some embodiments, the GI system of premature and/or newborn infants may be administered AOM preparations. For example, necrotizing enterocolitis may be treated or prevented in these subjects.

In accordance with one or more embodiments, AOM may be incorporated into various food and/or beverage products that are generally compatible therewith, for example, in terms of temperature, salt, alcohol, or preservative levels. In some embodiments, a culture of AOM may be applied, e.g., sprayed, onto a food or beverage prior to consumption. Some non-limiting examples of such food products include yogurt, milk, cheese, salads, fruit and bread. In terms of liquids containing AOM, upon consumption water may be absorbed through the stomach wall, and AOM may be filtered out and may be trapped in the mucus layer. Hot spots of ammonia release may be converted into hot spots of nitrite by the AOM. Acidified nitrite may serve as an anti-microbial agent, for example, preventing stomach infection. As stomach contents are passed down through the GI system, the mucus layers may be passed down too, and so the layer of mucus containing AOM may progress down the GI system for various beneficial results consistent with this disclosure.

Use of Microbiome Compatible Products with Administration of Ammonia Oxidizing Microorganisms

Microbiome compatible products may be used in conjunction with the preparations and methods disclosed herein. Various products may be considered to be “biome-friendly” or “biome-compatible.” Examples of biome-friendly products are disclosed in International (PCT) Patent Application Publication No. WO2017/004534 (International (PCT) Patent Application Serial No. PCT/US/2016/040723 as filed on Jul. 1, 2016) which is hereby incorporated herein by reference in its entirety for all purposes. Some biome-friendly products may be cosmetic or therapeutic in nature. In accordance with one or more embodiments, biome-friendly products may be used in combination with microorganisms, e.g., non-pathogenic microorganisms, e.g., ammonia oxidizing microorganisms, which may in turn be used in the form of a preparation or composition to be applied to a subject. Ammonia oxidizing compositions disclosed herein may be administered for a cosmetic or therapeutic indication in conjunction with a biome-friendly or biome-compatible product.

In accordance with one or more embodiments, a preparation, composition, formulation or product comprising ammonia oxidizing microorganisms, e.g., for cosmetic or therapeutic use, may itself be considered biome-friendly. In other embodiments, a preparation comprising ammonia oxidizing microorganisms may be used in conjunction with a biome-friendly product. In some embodiments, a preparation comprising ammonia oxidizing microorganisms may be mixed with a biome-friendly product or otherwise administered concurrently. In other embodiments, a preparation comprising ammonia oxidizing microorganisms may be distinct or separate from a biome-friendly product although potentially used in conjunction therewith. In some embodiments, a biome-friendly product is used alone. Ammonia oxidizing microorganism composition preparations for use in conjunction with a biome-friendly product may be formulated for cosmetic or therapeutic use.

Biome-friendly or biome-compatible products may be used in conjunction with an ammonia oxidizing microorganism preparation formulated for any mode of delivery, e.g., formulated for targeted delivery to a subject, e.g., to a target tissue, region, system, or organ of a subject. For example, the ammonia oxidizing microorganism preparation to be used in conjunction with a biome-friendly product may be formulated for delivery to the eye, ear, nose, urogenital system, respiratory system, or gastrointestinal system of the subject. In some embodiments, the ammonia oxidizing microorganism composition for use with a biome-friendly product may be formulated for targeted delivery based on a condition or disorder of a subject. For instance, the formulation for targeted delivery may be based on a desired local or systemic effect to be achieved, e.g., a local or systemic therapeutic or cosmetic effect.

Biome-friendly cosmetic products that may be used with the present disclosure may be, or include, or be disposed in any one or more of a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps, e.g., foaming body cleansers, and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

Products, e.g., microbiome-compatible cosmetic products, e.g., shampoos, conditioners, and cleansers, as described herein may be used in conjunction with the treatment of a condition, disease, or disorder. These cosmetic products may be used in conjunction with administration of the ammonia oxidizing microorganisms for therapeutic or cosmetic purposes. For example, throughout the treatment period or cosmetic period of time of administering the ammonia oxidizing bacteria to a subject, the microbiome-compatible cosmetic products may be used. The microbiome-compatible cosmetic products may be used for a period of time prior to commencement of treatment of the therapeutic or cosmetic condition through administration of ammonia oxidizing bacteria to a subject. The microbiome-compatible cosmetic products may be used for a period of time subsequent to commencement of treatment of the therapeutic or cosmetic condition through administration of ammonia oxidizing bacteria to a subject. The microbiome-compatible cosmetic products may be used for a period of time subsequent to discontinuation of therapeutic or cosmetic treatment of the condition through administration of ammonia oxidizing bacteria to a subject.

In some embodiments, the subject may apply one or more cosmetic product, and wait a period of time before administration of the ammonia oxidizing microorganisms. In other embodiments, the subject may administer the ammonia oxidizing microorganisms, and wait a period of time before applying one or more cosmetic products.

The period of time the subject may wait may be about 1 minute, 5 minutes, 10, 15, 20, 25, 30, 45, 60, 90, 120 minutes, or 3 hours, 4, 5, 6, 7, 8, 12, 18, 24 hours after applying one or more cosmetic product and prior to administration of ammonia oxidizing microorganisms.

The period of time the subject may wait may be about 1 minute, 5 minutes, 10, 15, 20, 25, 30, 45, 60, 90, 120 minutes, or 3 hours, 4, 5, 6, 7, 8, 12, 18, 24 hours after administering the ammonia oxidizing microorganisms and prior to applying one or more cosmetic products.

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

Certain embodiments are within the scope of the following claims.

1. A method of introducing ammonia oxidizing microorganisms (AOM) to a subject, comprising:
enterally administering a preparation comprising AOM to the subject.

2. The method of any of the preceding claims, wherein enteral administration comprises oral, buccal, sublabial, or sublingual administration.

3. A method of introducing AOM to a subject, comprising:
rectally administering a preparation comprising AOM to the subject.

4. The method of any of the preceding claims, wherein rectal administration comprises administration via suppository or enema.

5. The method of any of the preceding claims, wherein rectal administration is associated with a fecal microbiota transplant procedure.

6. A method of colonizing a gastrointestinal system of a subject with AOM, comprising:
administering an effective amount of a preparation comprising AOM to the gastrointestinal system of the subject,
wherein the AOM colonize a target tissue of the gastrointestinal system.

7. A method of improving digestion in a subject, comprising:
administering to the subject an effective amount of a preparation comprising AOM,
thereby improving digestion in the subject.

8. A method of treating a gastrointestinal disorder in a subject, comprising:
administering to the subject an effective amount of a preparation comprising AOM,
thereby treating the gastrointestinal disorder.

9. The method of any of the preceding claims, wherein an amount and/or a frequency of administration is sufficient to increase mucus thickness in at least a portion of the gastrointestinal system of the subject.

10. The method of any of the preceding claims, wherein the preparation comprising AOM is administered via ingestion to a gastrointestinal system of a subject.

11. The method of any of the preceding claims, wherein the subject has a substantially empty stomach when the preparation is administered.

12. The method of any of the preceding claims, wherein the preparation is administered subsequent to administration of an antibiotic or a cleansing bowel preparation.

13. The method of any of the preceding claims, wherein a target percentage of administered AOM are transferred to a gastrointestinal system of the subject.

14. The method of any of the preceding claims, further comprising administering water to the subject subsequent to administering the preparation.

15. The method of any of the preceding claims, wherein administering the preparation results in increased tolerance, decreased sensitivity, and/or improved uptake of nutrients in connection with a food or a beverage ingested by the subject.

16. The method of any of the preceding claims, wherein the gastrointestinal disorder is an inflammatory condition.

17. The method of any of the preceding claims, wherein the inflammatory condition is colitis, necrotizing enterocolitis, inflammatory bowel disease, ulcer, Crohn's Disease, ulcerative colitis, Celiac's Disease, gluten sensitivity, heartburn, pancreatitis, appendicitis, gastritis, gastroenteritis, irritable bowel syndrome, or a dental or periodontal condition.

18. The method of any of the preceding claims, wherein the gastrointestinal disorder relates to a lactose, food, or beverage intolerance, SIBO, a malabsorption disorder, a biliary disorder, reflux, or dispoxia.

19. The method of any of the preceding claims, wherein the inflammatory condition is associated with catheter-based delivery of a substance, e.g. enteral nutrition.

20. The method of any of the preceding claims, wherein administration precedes or follows a medical procedure, e.g., a catheterization, endoscopy, or colonoscopy procedure, or a dental procedure.

21. The method of any of the preceding claims, wherein the inflammatory condition is an infection by one or more of the following microorganisms: H. pylori, C. diff, cholera, amoebic dysentery, Y. enterocolitica, S. enteritidis, S. typhimurium, Shigella sonnei, and E. coli.

22. The method of any of the preceding claims, wherein the gastrointestinal disorder is characterized by constipation or diarrhea.

23. The method of any of the preceding claims, wherein the gastrointestinal disorder is characterized by hyperammonemia.

24. The method of any of the preceding claims, wherein the gastrointestinal disorder comprises liver failure, e.g. acute or chronic liver failure.

25. The method of any of the preceding claims, wherein a deposit tissue, target tissue, or both is a mucous membrane of the subject.

26. The method of any of the preceding claims, wherein a deposit tissue, target tissue, or both is associated with a stomach of the subject.

27. The method of any of the preceding claims, wherein a deposit tissue, target tissue, or both is a salivary gland, oral cavity, pharynx, tongue, esophagus, liver, gallbladder, common bile duct, colon (transverse, ascending, and/or descending), cecum, appendix, rectum, anus, pancreas, pancreatic duct, large intestine, or small intestine (duodenum, jejunum, and/or ileum) of the subject.

28. The method of any of the preceding claims, wherein the target tissue is associated with a desired local effect.

29. The method of any of the preceding claims, wherein the target tissue is associated with a desired systemic effect.

30. The method of any of the preceding claims, wherein the desired systemic effect involves treatment of one or more of: headaches, cardiovascular diseases, inflammation, immune responses, autoimmune disorders, liver diseases, infections, neurological diseases, psychiatric disorders, nitric oxide disorders, urea cycle disorders, congestion, vasodilation disorders, skin diseases, wound healing, reactions to insect bites, ophthalmic disorders, connective tissue disorders, and certain viral, bacterial, or fungal infections.

31. The method of any of the preceding claims, wherein administering an effective amount of the preparation promotes endothelial function.

32. The method of any of the preceding claims, wherein administering an effective amount of the preparation changes or alters a level of nitrite or NO at a target tissue or in circulation.

33. The method of any of the preceding claims, wherein administering an effective amount of the preparation modulates a microbiome associated with the gastrointestinal system of the subject.

34. The method of any of the preceding claims, wherein administering is device-assisted.

35. The method of any of the preceding claims, wherein the preparation is administered prior to onset of a gastrointestinal condition.

36. The method of any of the preceding claims, wherein the preparation is administered during incidence of a gastrointestinal condition.

37. The method of any of the preceding claims, wherein the preparation is administered subsequent to the subsiding of a gastrointestinal condition.

38. The method of any of the preceding claims, wherein the preparation is administered in response to a gastrointestinal symptom, trigger or warning sign, e.g. discomfort or a change in bowel habit.

39. The method of any of the preceding claims, further comprising determining whether the subject is in need of treatment for a gastrointestinal disorder.

40. The method of any of the preceding claims, wherein the preparation is administered as a solution, suspension, emulsion, ointment, gel, hydrogel, or liquid, e.g. drop, spray, aerosol, or mist.

41. The method of any of the preceding claims, wherein the preparation is formulated as a tablet or capsule.

42. The method of any of the preceding claims, wherein the preparation includes microspheres or microcapsules.

43. The method of any of the preceding claims, wherein the preparation is formulated to be compatible with the gastrointestinal system of the subject.

44. The method of any of the preceding claims, wherein administration reduces bloating, diarrhea, gas, stomach pain, stomach cramping, or borborygmus in the subject.

45. The method of any of the preceding claims, wherein the preparation is formulated for immediate release or extended release.

46. The method of any of the preceding claims, wherein the preparation is formulated to deliver nitrite or NO to a target tissue, locally or systemically.

47. The method of any of the preceding claims, wherein the preparation is formulated for transmucosal delivery and/or circulation, e.g. locally or systemically.

48. The method of any of the preceding claims, further comprising administering a second amount of the preparation to the subject.

49. The method of any of the preceding claims, wherein the preparation is administered as part of a combination therapy.

50. The method of any of the preceding claims, further comprising administering a second treatment in combination with the preparation.

51. The method of any of the preceding claims, wherein the preparation is administered for a period of time prior to initiating the second treatment.

52. The method of any of the preceding claims, wherein the preparation is administered concurrently with the second treatment.

53. The method of any of the preceding claims, wherein the preparation is administered for a period of time subsequent to ceasing the second treatment.

54. The method of any of the preceding claims, wherein the second treatment is administered via an alternate mode of administration, e.g. via inhalation or intranasal technique.

55. The method of any of the preceding claims, wherein the subject has a therapeutic level of a second treatment.

56. The method of any of the preceding claims, wherein the preparation is administered in conjunction with an anti-inflammatory agent.

57. The method of any of the preceding claims, wherein the preparation is administered in conjunction with a medical approach that treats, e.g., is approved to treat or is commonly used to treat, the relevant disease or disorder, or a symptom of the relevant disease or disorder.

58. The method of any of the preceding claims, wherein the preparation is administered before or after a surgical or diagnostic procedure.

59. The method of any of the preceding claims, wherein the preparation is administered in conjunction with exercise, fiber, laxative, antidiuretic, probiotic, therapeutic, or stress management.

60. The method of any of the preceding claims, wherein the preparation is administered in combination with a therapeutic treatment for colitis, necrotizing enterocolitis, inflammatory bowel disease, ulcer, Crohn's Disease, ulcerative colitis, Celiac's Disease, gluten sensitivity, heartburn, pancreatitis, appendicitis, gastritis, gastroenteritis, or irritable bowel syndrome.

61. The method of any of the preceding claims, wherein the preparation is administered in conjunction with nitrite, nitrate, and/or NO.

62. The method of any of the preceding claims, wherein the effective amount is a therapeutically effective dose of AOM.

63. The method of any of the preceding claims, wherein the therapeutically effective dose of AOM is about or greater than about 1×10³, 10⁴, 10⁵, 10⁶, 10⁷, 10⁸, 10⁹, 10¹⁰, 10¹¹, 10¹², 10¹³, or 10¹⁴ CFU.

64. The method of any of the preceding claims, wherein the preparation is administered as an analgesic.

65. The method of any of the preceding claims, wherein the preparation is administered as a prophylactic.

66. The method of any of the preceding claims, wherein the preparation is self-administered.

67. The method of any of the preceding claims, wherein the preparation is administered about 1, 2, 3, 4, 5, 6, 7, 8, 9, 10, 11, 12, 13, 14, 15, 16, 17, 18, 19, 20, 21, 22, 23, or 24 times per day.

68. The method of any of the preceding claims, wherein the preparation is administered for about 1-3, 3-5, 5-7, 7-9, 5-10, 10-14, 12-18, 12-21, 21-28, 28-35, 35-42, 42-49, 49-56, 46-63, 63-70, 70-77, 77-84, or 84-91 days.

69. The method of any of the preceding claims, wherein the preparation is administered within 30, 60, 90, 120, 150, or 180 minutes of the subject waking from sleep.

70. The method of any of the preceding claims, wherein the preparation is administered within 30, 60, 90, 120, 150, or 180 minutes prior to the subject sleeping.

71. The method of any of the preceding claims, wherein the preparation is administered within 30, 60, 90, 120, 150, or 180 minutes of the subject eating.

72. The method of any of the preceding claims, wherein the preparation is administered 30, 60, 90, 120, 150, or 180 minutes before the subject cleanses or showers.

73. The method of any of the preceding claims, wherein the subject is female.

74. The method of any of the preceding claims, wherein the subject is male.

75. The method of any of the preceding claims, wherein the subject is characterized as one of the following ethnicity/race: Asian, black or African American, Hispanic or Latino, white, or multi-racial.

76. The method of any of the preceding claims, wherein the subject has a disrupted microbiome.

77. The method of any of the preceding claims, wherein the subject is of an age less than 1, or between 1-5, 5-10, 10-20, 20-30, 30-40, 40-50, 50-60, or over 60 years.

78. The method of any of the preceding claims, wherein the preparation comprises AOM in a buffer solution, e.g., an aqueous buffer solution.

79. The method of any of the preceding claims, wherein the buffer solution, e.g., aqueous buffer solution, comprises disodium phosphate and magnesium chloride, for example, 50 mM Na₂HPO₄ and 2 mM MgCl₂ in water.

80. The method of any of the preceding claims, wherein the buffer solution e.g., aqueous buffer solution, consisting essentially of disodium phosphate and magnesium chloride, for example, 50 mM Na₂HPO₄ and 2 mM MgCl₂ in water.

81. The method of any of the preceding claims, wherein the buffer solution, e.g., aqueous buffer solution, consists of disodium phosphate and magnesium chloride, for example, 50 mM Na₂HPO₄ and 2 mM MgCl₂ in water.

82. The method of any of the preceding claims, wherein the preparation is characterized by a physiological pH level.

83. The method of any of the preceding claims, wherein the preparation further comprises or is administered concurrently with a compound that promotes growth or metabolism of the AOM, NO production, and/or urease activity.

84. The method of any of the preceding claims, wherein the preparation comprises at least one of ammonia, ammonium salts, and urea.

85. The method of any of the preceding claims, wherein the preparation comprises a controlled release material, e.g., slow release material.

86. The method of any of the preceding claims, wherein the preparation further comprises an excipient, e.g., a pharmaceutically acceptable excipient.

87. The method of any of the preceding claims, wherein the excipient comprises an absorption or penetration enhancer, preservative, antioxidant, buffer, chelating agent, ion exchange agent, solubilizing agent, suspending agent, thickener, surfactant, wetting agent, tonicity-adjusting agent, enzyme inhibitor, or vehicle for proper drug delivery.

88. The method of any of the preceding claims, wherein the preparation comprises a mucoadhesive agent.

89. The method of any of the preceding claims, wherein the preparation includes a disintegrant, chelator, coating agent, modified-release product, or filler.

90. The method of any of the preceding claims, wherein the preparation is substantially free of other organisms.

91. The method of any of the preceding claims, wherein the preparation comprises between about 1×10³ CFU/mL to about 1×10¹⁴ CFU/mL AOM.

92. The method of any of the preceding claims, wherein the preparation comprises between about 1×10⁹ CFU/mL to about 10×10⁹ CFU/mL AOM.

93. The method of any of the preceding claims, wherein the AOM comprise ammonia oxidizing bacteria (AOB).

94. The method of any of the preceding claims, wherein the AOM consist essentially of AOB.

95. The method of any of the preceding claims, wherein the AOM consist of AOB.

96. The method of any of the preceding claims, wherein the AOM comprise Nitrosomonas, Nitrosococcus, Nitrosospira, Nitrosocystis, Nitrosolobus, Nitrosovibrio, and combinations thereof.

97. The method of any of the preceding claims, wherein the AOM is Nitrosomonas eutropha (N. eutropha).

98. The method of any of the preceding claims, wherein the AOM is N. eutropha D23, having ATCC accession number PTA-121157.

99. The method of any of the preceding claims, wherein the AOM comprise ammonia oxidizing archaea (AOA).

100. The method of any of the preceding claims, wherein the AOM are capable of converting ammonia or ammonium to nitrite at a rate of at least about 1 pmol/min/mg protein, e.g., at least about 0.1 nmol/min/mg protein.

101. The method of any of the preceding claims, wherein the preparation is administered, e.g., via ingestion to a first tissue, e.g. a deposit tissue.

102. The method of any of the preceding claims, wherein the first tissue is the target tissue.

103. The method of any of the preceding claims, wherein the first tissue is other than the target tissue, e.g., the preparation is applied to a first tissue and the preparation, or a product of the preparation, e.g., NO, is transported, e.g., by diffusion, to a second tissue, e.g. the target tissue.

104. The method of any of the preceding claims, wherein the second treatment comprises a surgical procedure.

105. The method of any of the preceding claims, wherein the excipient comprises an anti-adherent, binder, coat, disintegrant, filler, flavor, color, lubricant, glidant, sorbent preservative, or sweetener.

106. The method of any of the preceding claims, wherein a biome-friendly product is used in connection with the administered preparation comprising AOM.

107. A preparation comprising AOM, as recited in any of the preceding claims, for enteral administration to a subject.

108. The preparation of any of the preceding claims, wherein the preparation is a food product.

109. The preparation of any of the preceding claims, wherein the food product comprises a food, drink, supplement, nutraceutical, additive, or medical nutritional product.

110. A preparation comprising AOM, as recited in any of the preceding claims, for treatment of a gastrointestinal disorder in a subject.

111. The preparation of any of the preceding claims, wherein the preparation is packaged for single use.

112. The preparation of any of the preceding claims, wherein the preparation is packaged for multiple use.

113. The preparation of any of the preceding claims, comprising AOM and other organisms, e.g., a community of organisms.

114. A device configured to administer a preparation comprising AOM, as recited in any of the preceding claims, to a target tissue of a gastrointestinal system of a subject.

115. A kit comprising a preparation comprising AOM as recited in any of the preceding claims.
